SEQUENCE LISTING (73 sequences)

SEQ ID NO: 1 - protein, 5 aa, Artificial Sequence
  heavy chain CDR1 of AbF46
DYYMS

SEQ ID NO: 2 - protein, 19 aa, Artificial Sequence
  heavy chain CDR2 of AbF46
FIRNKANGYTTEYSASVKG

SEQ ID NO: 3 - protein, 6 aa, Artificial Sequence
  heavy chain CDR3 of AbF46
DNWFAY

SEQ ID NO: 4 - protein, 6 aa, Artificial Sequence
  heavy chain CDR1 of c-Met antibody
XXYYMS

SEQ ID NO: 5 - protein, 8 aa, Artificial Sequence
  heavy chain CDR2 of c-Met antibody
ANXXNGXT

SEQ ID NO: 6 - protein, 6 aa, Artificial Sequence
  heavy chain CDR3 of c-Met antibody
DNWLXY

SEQ ID NO: 7 - protein, 17 aa, Artificial Sequence
  light chain CDR1 of c-Met antibody
KSSXSLLAXGNXXNYLA

SEQ ID NO: 8 - protein, 7 aa, Artificial Sequence
  light chain CDR2 of c-Met antibody
WXSXRVX

SEQ ID NO: 9 - protein, 9 aa, Artificial Sequence
  light chain CDR3 of c-Met antibody
XQSYSXPXT

SEQ ID NO: 10 - protein, 17 aa, Artificial Sequence
  light chain CDR1 of AbF46
KSSQSLLASGNQNNYLA

SEQ ID NO: 11 - protein, 7 aa, Artificial Sequence
  light chain CDR2 of AbF46
WASTRVS

SEQ ID NO: 12 - protein, 9 aa, Artificial Sequence
  light chain CDR3 of AbF46
QQSYSAPLT

SEQ ID NO: 13 - protein, 9 aa, Artificial Sequence
  CDR-L3 derived from L3-1 clone
QQSYSRPYT

SEQ ID NO: 14 - protein, 9 aa, Artificial Sequence
  CDR-L3 derived from L3-2 clone
GQSYSRPLT

SEQ ID NO: 15 - protein, 9 aa, Artificial Sequence
  CDR-L3 derived from L3-3 clone
AQSYSHPFS

SEQ ID NO: 16 - protein, 9 aa, Artificial Sequence
  CDR-L3 derived from L3-5 clone
QQSYSRPFT

SEQ ID NO: 17 - protein, 117 aa, Artificial Sequence
  heavy chain variable region of anti c-Met humanized antibody(huAbF46-H4)
EVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSS

SEQ ID NO: 18 - protein, 114 aa, Artificial Sequence
  light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPYTFGQGTKVEIKR

SEQ ID NO: 19 - protein, 114 aa, Artificial Sequence
  light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCGQSYSRPLTFGQGTKVEIKR

SEQ ID NO: 20 - protein, 114 aa, Artificial Sequence
  light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCAQSYSHPFSFGQGTKVEIKR

SEQ ID NO: 21 - protein, 114 aa, Artificial Sequence
  light chain variable region of anti c-Met humanized antibody(huAbF46-H4)
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSRPFTFGQGTKVEIKR

SEQ ID NO: 22 - protein, 6 aa, Artificial Sequence
  CDR-H1 derived from H11-4 clone
PEYYMS

SEQ ID NO: 23 - protein, 6 aa, Artificial Sequence
  CDR-H1 derived from YC151 clone
PDYYMS

SEQ ID NO: 24 - protein, 6 aa, Artificial Sequence
  CDR-H1 derived from YC193 clone
SDYYMS

SEQ ID NO: 25 - protein, 8 aa, Artificial Sequence
  CDR-H2 derived from YC244 clone
RNNANGNT

SEQ ID NO: 26 - protein, 8 aa, Artificial Sequence
  CDR-H2 derived from YC321 clone
RNKVNGYT

SEQ ID NO: 27 - protein, 6 aa, Artificial Sequence
  CDR-H3 derived from YC354 clone
DNWLSY

SEQ ID NO: 28 - protein, 6 aa, Artificial Sequence
  CDR-H3 derived from YC374 clone
DNWLTY

SEQ ID NO: 29 - protein, 17 aa, Artificial Sequence
  CDR-L1 derived from L1-1 clone
KSSHSLLASGNQNNYLA

SEQ ID NO: 30 - protein, 17 aa, Artificial Sequence
  CDR-L1 derived from L1-3 clone
KSSRSLLSSGNHKNYLA

SEQ ID NO: 31 - protein, 17 aa, Artificial Sequence
  CDR-L1 derived from L1-4 clone
KSSKSLLASGNQNNYLA

SEQ ID NO: 32 - protein, 17 aa, Artificial Sequence
  CDR-L1 derived from L1-12 clone
KSSRSLLASGNQNNYLA

SEQ ID NO: 33 - protein, 17 aa, Artificial Sequence
  CDR-L1 derived from L1-22 clone
KSSHSLLASGNQNNYLA

SEQ ID NO: 34 - protein, 7 aa, Artificial Sequence
  CDR-L2 derived from L2-9 clone
WASKRVS

SEQ ID NO: 35 - protein, 7 aa, Artificial Sequence
  CDR-L2 derived from L2-12 clone
WGSTRVS

SEQ ID NO: 36 - protein, 7 aa, Artificial Sequence
  CDR-L2 derived from L2-16 clone
WGSTRVP

SEQ ID NO: 37 - protein, 9 aa, Artificial Sequence
  CDR-L3 derived from L3-32 clone
QQSYSKPFT

SEQ ID NO: 38 - DNA, 1416 bp, Artificial Sequence
  nucleotide sequence of heavy chain of chAbF46
gaattcgccgccaccatggaatggagctgggtttttctcgtaacacttttaaatggtatccagtgtgaggtgaagctggtggagtctggaggaggcttggtacagcctgggggttctctgagactctcctgtgcaacttctgggttcaccttcactgattactacatgagctgggtccgccagcctccaggaaaggcacttgagtggttgggttttattagaaacaaagctaatggttacacaacagagtacagtgcatctgtgaagggtcggttcaccatctccagagataattcccaaagcatcctctatcttcaaatggacaccctgagagctgaggacagtgccacttattactgtgcaagagataactggtttgcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 39 - DNA, 759 bp, Artificial Sequence
  nucleotide sequence of light chain of chAbF46
gaattcactagtgattaattcgccgccaccatggattcacaggcccaggtcctcatgttgctgctgctatcggtatctggtacctgtggagacattttgatgacccagtctccatcctccctgactgtgtcagcaggagagaaggtcactatgagctgcaagtccagtcagagtcttttagctagtggcaaccaaaataactacttggcctggcaccagcagaaaccaggacgatctcctaaaatgctgataatttgggcatccactagggtatctggagtccctgatcgcttcataggcagtggatctgggacggatttcactctgaccatcaacagtgtgcaggctgaagatctggctgtttattactgtcagcagtcctacagcgctccgctcacgttcggtgctgggaccaagctggagctgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 40 - protein, 447 aa, Artificial Sequence
  amino acid sequence of H1-heavy
EVQLVESGGGLVQPGGSLRLSCAASGFTFTDYYMSWVRQAPGKGLEWLGFIRNKANGYTTEYSASVKGRFTISRDNSKNSLYLQMNSLKTEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 447 aa, Artificial Sequence
  amino acid sequence of H3-heavy
EVQLVESGGGLVQPGGSLRLSCAASGFTFTDYYMSWVRQAPGKGLEWLGFIRNKANGYTTEYSASVKGRFTISRDNSKNSLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 447 aa, Artificial Sequence
  amino acid sequence of H4-heavy
EVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 220 aa, Artificial Sequence
  amino acid sequence of H1-light
DIVMTQSPDSLAVSLGERATINCKSSQSLLASGNQNNYLAWHQQKPGQPPKMLIIWASTRVSGVPDRFSGSGSGTDFTLTISSLQAEDVAVYYCQQSYSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 220 aa, Artificial Sequence
  amino acid sequence of H2-light
DIVMTQTPLSLPVTPGEPASISCKSSQSLLASGNQNNYLAWHLQKPGQSPQMLIIWASTRVSGVPDRFSGSGSGTDFTLKISRVEAEDVGVYYCQQSYSAPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 220 aa, Artificial Sequence
  amino acid sequence of H3-light
DIVMTQSPDSLAVSLGERATINCKSSQSLLASGNQNNYLAWYQQKPGQPPKLLIIWASTRVSGVPDRFSGSGSGTDFTLTISSLQAEDVAVYYCQQSYSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 219 aa, Artificial Sequence
  amino acid sequence of H4-light
DIQMTQSPSSLSASVGDRVTITCKSSQSLLASGNQNNYLAWHQQKPGKAPKMLIIWASTRVSGVPSRFSGSGSGTDFTLTISSLQPEDFATYYCQQSYSAPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 47 - DNA, 1350 bp, Artificial Sequence
  nucleotide sequence of H1-heavy
gaggtgcagctggtggagtctgggggaggcttggtccagcctggagggtccctgagactctcctgtgcagcctctggattcaccttcactgactactacatgagctgggtccgccaggctccagggaaggggctggagtggttgggctttattagaaacaaagctaacggttacaccacagaatacagtgcgtctgtgaaaggcagattcaccatctcaagagataattcaaagaactcactgtatctgcaaatgaacagcctgaaaaccgaggacacggccgtgtattactgtgctagagataactggtttgcttactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 48 - DNA, 1350 bp, Artificial Sequence
  nucleotide sequence of H3-heavy
gaggtgcagctggtggagtctgggggaggcttggtccagcctggagggtccctgagactctcctgtgcagcctctggattcaccttcactgactactacatgagctgggtccgccaggctccagggaaggggctggagtggttgggctttattagaaacaaagctaacggttacaccacagaatacagtgcgtctgtgaaaggcagattcaccatctcaagagataattcaaagaactcactgtatctgcaaatgaacagcctgcgtgctgaggacacggccgtgtattactgtgctagagataactggtttgcttactggggtcaaggaaccctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 49 - DNA, 1350 bp, Artificial Sequence
  nucleotide sequence of H4-heavy
gaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 50 - DNA, 669 bp, Artificial Sequence
  nucleotide sequence of H1-light
gacatcgtgatgacccagtctccagactccctggctgtgtctctgggcgagagggccaccatcaactgcaagtccagccagagtcttttagctagcggcaaccaaaataactacttagcttggcaccagcagaaaccaggacagcctcctaagatgctcattatttgggcatctacccgggtatccggggtccctgaccgattcagtggcagcgggtctgggacagatttcactctcaccatcagcagcctgcaggctgaagatgtggcagtttattactgtcagcaatcctatagtgctcctctcacgttcggaggcggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 51 - DNA, 669 bp, Artificial Sequence
  nucleotide sequence of H2-light
gatattgtgatgacccagactccactctccctgcccgtcacccctggagagccggcctccatctcctgcaagtccagtcagagtcttttagctagtggcaaccaaaataactacttggcctggcacctgcagaagccagggcagtctccacagatgctgatcatttgggcatccactagggtatctggagtcccagacaggttcagtggcagtgggtcaggcactgatttcacactgaaaatcagcagggtggaggctgaggatgttggagtttattactgccagcagtcctacagcgctccgctcacgttcggacagggtaccaagctggagctcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 52 - DNA, 669 bp, Artificial Sequence
  nucleotide sequence of H3-light
gacatcgtgatgacccagtctccagactccctggctgtgtctctgggcgagagggccaccatcaactgcaagtccagccagagtcttttagctagcggcaaccaaaataactacttagcttggtaccagcagaaaccaggacagcctcctaagctgctcattatttgggcatctacccgggtatccggggtccctgaccgattcagtggcagcgggtctgggacagatttcactctcaccatcagcagcctgcaggctgaagatgtggcagtttattactgtcagcaatcctatagtgctcctctcacgttcggaggcggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 53 - DNA, 669 bp, Artificial Sequence
  nucleotide sequence of H4-light
gatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagcgctccgctcacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 54 - protein, 23 aa, Artificial Sequence
  linker between VH and VL
GLGGLGGGGSGGGGSGGSSGVGS

SEQ ID NO: 55 - DNA, 1088 bp, Artificial Sequence
  polynucleotide encoding scFv of huAbF46 antibody
gctagcgttttagcagaagttcaattggttgaatctggtggtggtttggttcaaccaggtggttctttgagattgtcttgtgctgcttctggttttactttcaccgattattacatgtcctgggttagacaagctccaggtaaaggtttggaatggttgggtttcattagaaacaaggctaacggttacactaccgaatattctgcttctgttaagggtagattcaccatttctagagacaactctaagaacaccttgtacttgcaaatgaactccttgagagctgaagatactgctgtttattactgcgctagagataattggtttgcttattggggtcaaggtactttggttactgtttcttctggcctcgggggcctcggaggaggaggtagtggcggaggaggctccggtggatccagcggtgtgggttccgatattcaaatgacccaatctccatcttctttgtctgcttcagttggtgatagagttaccattacttgtaagtcctcccaatctttgttggcttctggtaatcagaacaattacttggcttggcatcaacaaaaaccaggtaaagctccaaagatgttgattatttgggcttctaccagagtttctggtgttccatctagattttctggttctggttccggtactgattttactttgaccatttcatccttgcaaccagaagatttcgctacttactactgtcaacaatcttactctgctccattgacttttggtcaaggtacaaaggtcgaaatcaagagagaattcggtaagcctatccctaaccctctcctcggtctcgattctacgggtggtggtggatctggtggtggtggttctggtggtggtggttctcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttgagtttaaac

SEQ ID NO: 56 - DNA, 5597 bp, Artificial Sequence
  expression vector including polynucleotide encoding scFv of huAbF46 antibody
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagctaattctacttcatacattttcaattaagatgcagttacttcgctgtttttcaatattttctgttattgctagcgttttagcagaagttcaattggttgaatctggtggtggtttggttcaaccaggtggttctttgagattgtcttgtgctgcttctggttttactttcaccgattattacatgtcctgggttagacaagctccaggtaaaggtttggaatggttgggtttcattagaaacaaggctaacggttacactaccgaatattctgcttctgttaagggtagattcaccatttctagagacaactctaagaacaccttgtacttgcaaatgaactccttgagagctgaagatactgctgtttattactgcgctagagataattggtttgcttattggggtcaaggtactttggttactgtttcttctggcctcgggggcctcggaggaggaggtagtggcggaggaggctccggtggatccagcggtgtgggttccgatattcaaatgacccaatctccatcttctttgtctgcttcagttggtgatagagttaccattacttgtaagtcctcccaatctttgttggcttctggtaatcagaacaattacttggcttggcatcaacaaaaaccaggtaaagctccaaagatgttgattatttgggcttctaccagagtttctggtgttccatctagattttctggttctggttccggtactgattttactttgaccatttcatccttgcaaccagaagatttcgctacttactactgtcaacaatcttactctgctccattgacttttggtcaaggtacaaaggtcgaaatcaagagagaattcggtaagcctatccctaaccctctcctcggtctcgattctacgggtggtggtggatctggtggtggtggttctggtggtggtggttctcaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttgagtttaaacccgctgatctgataacaacagtgtagatgtaacaaaatcgactttgttcccactgtacttttagctcgtacaaaatacaatatacttttcatttctccgtaaacaacatgttttcccatgtaatatccttttctatttttcgttccgttaccaactttacacatactttatatagctattcacttctatacactaaaaaactaagacaattttaattttgctgcctgccatatttcaatttgttataaattcctataatttatcctattagtagctaaaaaaagatgaatgtgaatcgaatcctaagagaattgggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctctcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccgtgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaatttcttgaagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctggctagt

SEQ ID NO: 57 - protein, 13 aa, Artificial Sequence
  U6-HC7 hinge
EPKSCDCHCPPCP

SEQ ID NO: 58 - DNA, 435 bp, Artificial Sequence
  polynucleotide encoding CDR-L3 derived from L3-1 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtacacgttcggacagggtaccaaggtggagatcaaacgtacg

SEQ ID NO: 59 - DNA, 435 bp, Artificial Sequence
  polynucleotide encoding CDR-L3 derived from L3-2 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtgggcagtcctacagccgtccgctcacgttcggacagggtaccaaggtggagatcaaacgtacg

SEQ ID NO: 60 - DNA, 435 bp, Artificial Sequence
  polynucleotide encoding CDR-L3 derived from L3-3 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtgcacagtcctacagccatccgttctctttcggacagggtaccaaggtggagatcaaacgtacg

SEQ ID NO: 61 - DNA, 435 bp, Artificial Sequence
  polynucleotide encoding CDR-L3 derived from L3-5 clone
gaattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggcaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtttacgttcggacagggtaccaaggtggagatcaaacgtacg

SEQ ID NO: 62 - protein, 462 aa, Artificial Sequence
  polypeptide consisting of heavy chain of huAbF46-H4-A1, U6-HC7  hinge and constant region of human IgG1
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - DNA, 1410 bp, Artificial Sequence
  polynucleotide encoding polypeptide consisting of heavy chain of huAbF46-H4-A1, U6-HC7 hinge and constant region of human IgG1
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaaagctgcgattgccactgtcctccatgtccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 64 - protein, 461 aa, Artificial Sequence
  polypeptide consisting of heavy chain of huAbF46-H4-A1, human IgG2 hinge and constant region of human IgG1
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 1407 bp, Artificial Sequence
  polynucleotide encoding polypeptide consisting of heavy chain of huAbF46-H4-A1, human IgG2 hinge and constant region of human IgG1
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagaggaagtgctgtgtggagtgccccccctgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 66 - protein, 460 aa, Artificial Sequence
  polypeptide consisting of heavy chain of huAbF46-H4-A1, human IgG2 hinge and constant region of human IgG2
MEWSWVFLVTLLNGIQCEVQLVESGGGLVQPGGSLRLSCAASGFTFTDYYMSWVRQAPGKGLEWLGFIRNKANGYTTEYSASVKGRFTISRDNSKNTLYLQMNSLRAEDTAVYYCARDNWFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - DNA, 1404 bp, Artificial Sequence
  polynucleotide encoding polypeptide consisting of heavy chain of huAbF46-H4-A1, human IgG2 hinge and constant region of human IgG2
gaattcgccgccaccatggaatggagctgggtttttctcgtaacacttttaaatggtatccagtgtgaggttcagctggtggagtctggcggtggcctggtgcagccagggggctcactccgtttgtcctgtgcagcttctggcttcaccttcactgattactacatgagctgggtgcgtcaggccccgggtaagggcctggaatggttgggttttattagaaacaaagctaatggttacacaacagagtacagtgcatctgtgaagggtcgtttcactataagcagagataattccaaaaacacactgtacctgcagatgaacagcctgcgtgctgaggacactgccgtctattattgtgctagagataactggtttgcttactggggccaagggactctggtcaccgtctcctcggctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgag

SEQ ID NO: 68 - protein, 240 aa, Artificial Sequence [sequence omitted]

SEQ ID NO: 69 - DNA, 758 bp, Artificial Sequence
  polynucleotide encoding polypeptide consisting of light chain of huAbF46-H4-A1(H36Y) and human kappa constant region
aattcactagtgattaattcgccgccaccatggattcacaggcccaggtcctcatgttgctgctgctatcggtatctggtacctgtggagatatccagatgacccagtccccgagctccctgtccgcctctgtgggcgatagggtcaccatcacctgcaagtccagtcagagtcttttagctagtggcaaccaaaataactacttggcctggtaccaacagaaaccaggaaaagctccgaaaatgctgattatttgggcatccactagggtatctggagtcccttctcgcttctctggatccgggtctgggacggatttcactctgaccatcagcagtctgcagccggaagacttcgcaacttattactgtcagcagtcctacagccgcccgtacacgttcggacagggtaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgactcgag

SEQ ID NO: 70 - protein, 240 aa, Artificial Sequence
  polypeptide consisting of light chain of huAbF46-H4-A1 and human kappa constant region
MDSQAQVLMLLLLSVSGTCGDIQMTQSPSSLSASVGDRVTITCKSSQSLLASGNQNNHLAWYQQKPGKAPKMLIIWASTRVSGVPSRFSGSGSGTDFTLTISSLQPEDFATYYCQQSYSRPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 17 aa, Artificial Sequence
  CDR-L1 of antibody L3-11Y
KSSQSLLAWGNQNNYLA

SEQ ID NO: 72 - protein, 114 aa, Artificial Sequence
  amino acid sequence of light chain variable region of antibody L3-11Y
DIQMTQSPSSLSASVGDRVTITCKSSQSLLAWGNQNNYLAWYQQKPGKAPKMLIIWASTRVSGVPSRFSGSGSGTDFTLTISSLQPEDFATYYCQQSYSRPYTFGQGTKVEIKR

SEQ ID NO: 73 - protein, 220 aa, Artificial Sequence
  amino acid sequence of light chain of antibody L3-11Y
DIQMTQSPSSLSASVGDRVTITCKSSQSLLAWGNQNNYLAWYQQKPGKAPKMLIIWASTRVSGVPSRFSGSGSGTDFTLTISSLQPEDFATYYCQQSYSRPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2014/0154251 A1
(43) Pub. Date: Jun. 5, 2014

(54) ANTI C-MET ANTIBODY AND USES THEREOF

(71) Applicant: Samsung Electronics Co., Ltd., Suwon-si (KR)

(72) Inventors: Seung Hyun LEE, Suwon-si (KR); Geun Woong Kim, Yongin-si (KR); Kyung Ah Kim, Seongnam-si (KR); Hye Won Park, Pyeongtaek-si (KR); Ho Yeong Song, Seongnam-si (KR); Young Mi Oh, Incheon (KR); Saet Byoul Lee, Seoul (KR); Ji Min Lee, Seoul (KR); Kwang Ho Cheong, Seoul (KR); Yun Ju Jeong, Hwaseong-si (KR); Mi Young Cho, Seoul (KR); Jae Hyun Choi, Seongnam-si (KR); Yun Jeong Song, Seongnam-si (KR); Yoon Aa Choi, Busan (KR)

(73) Assignee: Samsung Electronics Co., Ltd., Suwon-si (KR)

(21) Appl. No.: 14/081,917

(22) Filed: Nov. 15, 2013

Related U.S. Application Data
(63) Continuation-in-part of application No. 13/646,589, filed on Oct. 5, 2012.

(30) Foreign Application Priority Data
Oct. 5, 2011 (KR) 10-2011-0101293
Jul. 9, 2012 (KR) 10-2012-0074691
Oct. 5, 2012 (KR) 10-2012-0110584

(51) Int. Cl. C07K 16/28

(52) U.S. Cl. CPC C07K 16/2863
USPC 424/135.1; 424/133.1; 424/143.1; 424/172.1

(57) ABSTRACT

An anti-c-Met antibody or antibody fragment and pharmaceutical composition comprising same, as well as a method for preventing and treating cancer by administering the antibody to a subject are provided.

### CROSS-REFERENCE TO RELATED APPLICATIONS

This application is a continuation-in-part (CIP) of co-pending U.S. patent application Ser. No. 13/646,589, which claims the benefit of Korean Patent Application No. 10-2011-0101293, filed on Oct. 5, 2011, Korean Patent Application No. 10-2012-0074691, filed on Jul. 9, 2012, and Korean Patent Application No. 10-2012-0110584, filed on Oct. 5, 2012, in the Korean Intellectual Property Office, the disclosures of which are herein incorporated by reference in their entirety.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows:—One 88,355 bytes ASCII (Text) file named “714350 sequencelisting.txt,” created Nov. 14, 2013.

### BACKGROUND

1. Field

The present disclosure relates to an anti-c-Met antibody and a pharmaceutical composition comprising the same for preventing and treating cancer.

2. Description of the Related Art

c-Met is a receptor for hepatocyte growth factor (HGF), a cytokine that binds the extracellular region of the c-Met receptor tyrosine kinase to induce cell division, movement, morphogenesis, and angiogenesis of various normal cells and tumor cells. c-Met is a representative receptor tyrosine kinase existing on the surface of cells, is itself a proto-oncogene, and is sometimes involved in various mechanisms related to cancer, such as cancer development, metastasis, migration, invasion, and angiogenesis, independent from a ligand, HGF. Thus, c-Met has been recently emerging as a new target for anti-cancer therapy.

In particular, c-Met is known to be involved in induction of resistance to commonly used anti-cancer drugs, and thus is regarded as important with respect to personalized treatments. Representative anti-cancer therapeutic drugs targeting epidermal growth factor receptor EGFR (ERBB1), i.e., Eribitux or Tarceva, work by blocking the signaling related to cancer development. In addition, Herceptin, which is well known as a breast cancer therapeutic drug, targets ERBB2 (HER2) and works by blocking the transduction of signals necessary for cell proliferation. Among patients resistant to the drugs described above, the signal transduction pathway that induces cell proliferation is not blocked due to the overexpression of c-Met. Thus, c-met has emerged as a target of interest for many pharmaceutical companies. Still, there is a need for additional anti-c-Met antibodies and related methods and compositions.

### SUMMARY

Provided is an anti-c-Met antibody or an antigen-binding fragment thereof. In one aspect, the anti-c-Met antibody or antigen-binding fragment thereof comprises a heavy chain variable region comprising at least one heavy chain complementarity determining region (CDR) selected from the group consisting of CDR-H1 having an amino acid sequence of SEQ ID NO: 4, CDR-H2 having an amino acid sequence of SEQ ID NO: 5, and CDR-H3 having an amino acid sequence of SEQ ID NO: 6; and a light chain variable region comprising at least one light chain CDR selected from the group consisting of CDR-L1 having an amino acid sequence of SEQ ID NO: 7, CDR-L2 having an amino acid sequence of SEQ ID NO: 8, and CDR-L3 having an amino acid sequence of SEQ ID NO: 9, wherein SEQ ID NOs: 4 to 9 are respectively represented by Formulas I to VI, described herein. In another aspect, the anti-c-Met antibody or antigen-binding fragment thereof comprises a heavy chain variable region comprising at least one heavy chain complementarity determining region (CDR) selected from the group consisting of CDR-H1 having an amino acid sequence of SEQ ID NO: 1, CDR-H2 having an amino acid sequence of SEQ ID NO: 2, and CDR-H3 having an amino acid sequence of SEQ ID NO: 3; and a light chain variable region comprising at least one light chain CDR selected from the group consisting of CDR-L1 having an amino acid sequence of SEQ ID NO: 7, CDR-L2 having an amino acid sequence of SEQ ID NO: 8, and CDR-L3 having an amino acid sequence of SEQ ID NO: 9, wherein SEQ ID NOS: 7 to 9 are respectively represented by Formulas IV to VI described herein. Nucleic acids encoding the antibodies and antibody fragments also are provided.

Further provided is a pharmaceutical composition including an anti-c-Met antibody or an antigen-binding fragment thereof, a method for preventing or treating cancer by administering the antibody or antigen-binding fragment thereof, as well as related methods and compositions.

### BRIEF DESCRIPTION OF THE DRAWINGS

These and/or other aspects will become apparent and more readily appreciated from the following description of the embodiments, taken in conjunction with the accompanying drawings of which:

FIG. 1 is a diagram showing the use of overlap extension PCR to obtain a scFv gene library of huAbF46 antibodies in which a desired CDR is mutated;

FIG. 2 is a graph of BrdU (%) plotted against antibody concentration, showing c-Met agonistic effect of huAbF46-H4-A1, huAbF46-H4-A2, huAbF46-H4-A3, and huAbF46-H4-A5 antibodies in a BrdU assay;

FIG. 3 is a graph of relative cell viability (%) plotted against antibody concentration, illustrating of the effect of huAbF46-H4-A1, huAbF46-H4-A2, huAbF46-H4-A3, and huAbF46-H4-A5 antibodies on in vitro cell proliferation;

FIG. 4 is a graph of Akt phosphorylation (%) plotted against treatment antibody, which shows the degree of agonism of huAbF46-H4-A1, huAbF46-H4-A2, huAbF46-H4-A3, and huAbF46-H4-A5 antibodies;

FIG. 5 is a graph illustrating anti-cancer effects of huAbF46-H4-A1, huAbF46-H4-A2, huAbF46-H4-A3, and huAbF46-H4-A5 antibodies as measured by the degree of degradation of c-Met;

FIGS. 6A and 6B are graphs of tumor volume plotted against time (days), showing in vivo anti-cancer effects of various concentrations of huAbF46-H4-A1 antibody in U87MG brain cancer mouse xenograft model or MKN45 gastric cancer mouse xenograft model;

FIG. 7 is a graph of relative cell viability (%) plotted against antibody concentration, showing the effect of huAbF46-H4-A1 (U6-HC7), huAbF46-H4-A1 (IgG2 hinge), and huAbF46-H4-A1 (IgG2 Fc) antibodies on in vitro cell proliferation;

FIGS. 8A and 8B are graphs of Akt phosphorylation (%) plotted against treatment antibody, which shows the degree of agonism of the antibodies. FIG. 8A shows the degree of Akt phosphorylation by huAbF46-H4-A1 (U6-HC7), huAbF46-H4-A1 (IgG2 hinge), and huAbF46-H4-A1 (IgG2 Fc) antibodies, and FIG. 8B shows the degree of Akt phosphorylation by huAbF46-H4-A1 (IgG2 Fc) and L3-11Y antibodies;

FIGS. 9A and 9B are graphs illustrating anti-cancer effects of antibodies as measured by degree of degradation of c-Met. FIG. 9A shows the degree of degradation of c-Met by huAbF46-H4-A1 (U6-HC7), huAbF46-H4-A1 (IgG2 hinge), and huAbF46-H4-A1 (IgG2 Fc) antibodies, and FIG. 9B shows the degree of degradation of c-Met by huAbF46-H4-A1 (IgG2 Fc) and L3-11Y antibodies; and

FIGS. 10A and 10B are graphs of tumor volume plotted against time (days), showing in vivo anti-cancer effects of huAbF46-H4-A1 (U6-HC7), huAbF46-H4-A1 (IgG2 hinge), and huAbF46-H4-A1 (IgG2 Fc) antibodies in U87MG brain cancer mouse xenograft model or MKN45 gastric cancer mouse xenograft model.

FIG. 11 is a graph of tumor volume plotted against time (days), showing dose-dependent in vivo anti-cancer effect of huAbF46-H4-A1 (IgG2 Fc) in EBC1 lung cancer mouse xenograft model.

FIG. 12 is a graph of tumor volume plotted against time (days), showing another example of in vivo anti-cancer effect of huAbF46-H4-A1 (IgG2 Fc) in EBC1 lung cancer mouse xenograft model.

FIG. 13 is a graph of tumor volume plotted against time (days), showing in vivo anti-cancer effect of huAbF46-H4-A1 (IgG2 Fc) in MHCC97H liver cancer model.

FIGS. 14 and 15 are graphs of tumor volume plotted against time (days), showing in vivo anti-cancer effect of huAbF46-H4-A1 (IgG2 Fc) in PDT (patient-derived tumor) xenograft models.

FIG. 14 shows the results for a NSCLC PDT, and

FIG. 15 shows the results for an RCC PDT.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one” when preceding a list of elements, modify the entire list of elements and do not modify the individual elements of the list.

According to an embodiment, there is provided an anti-c-Met antibody or an antigen-binding fragment thereof, wherein the antibody includes: a heavy chain variable region having the amino acid sequence of at least one heavy chain complementarity determining region (CDR) selected from the group consisting of CDR-H1 having an amino acid sequence of SEQ ID NO: 4, CDR-H2 having an amino acid sequence of SEQ ID NO: 5, and CDR-H3 having an amino acid sequence of SEQ ID NO: 6; and a light chain variable region having the amino acid sequence of at least one light chain complementarity determining region selected from the group consisting of CDR-L1 having an amino acid sequence of SEQ ID NO: 7, CDR-L2 having an amino acid sequence of SEQ ID NO: 8, and CDR-L3 having an amino acid sequence of SEQ ID NO: 9, in which SEQ ID NOS: 4 to 9 are respectively represented by Formula I to VI below:

Formula I
(SEQ ID NO: 4)
Xaa₁-Xaa₂-Tyr-Tyr-Met-Ser
Formula II
(SEQ ID NO: 5)
Arg-Asn-Xaa₃-Xaa₄-Asn-Gly-Xaa₅-Thr
Formula III
(SEQ ID NO: 6)
Asp-Asn-Trp-Leu-Xaa₆-Tyr
Formula IV
(SEQ ID NO: 7)
Lys-Ser-Ser-Xaa₇-Ser-Leu-Leu-Ala-Xaa₈-Gly-
Asn-Xaa₉-Xaa₁₀-Asn-Tyr-Leu-Ala
Formula V
(SEQ ID NO: 8)
Trp-Xaa₁₁-Ser-Xaa₁₂-Arg-Val-Xaa₁₃
Formula VI
(SEQ ID NO: 9)
Xaa₁₄-Gln-Ser-Tyr-Ser-Xaa₁₅-Pro-Xaa₁₆-Thr

In Formula I, Xaa₁ is Pro or Ser or absent, and Xaa₂ is Glu or Asp.

In Formula II, Xaa₃ is Asn or Lys, Xaa₄ is Ala or Val, and Xaa₅ is Asn or Thr.

In Formula III, Xaa₆ is Ser or Thr.

In Formula IV, Xaa₇ is His, Arg, Gln, or Lys, Xaa₈ is Ser or Trp, Xaa₉ is His or Gln, and Xaa₁₀ is Lys or Asn.

In Formula V, Xaa₁₁ is Ala or Gly, Xaa₁₂ is Thr or Lys, and Xaa₁₃ is Ser or Pro.

In Formula VI, Xaa₁₄ is Gly, Ala, or Gln, Xaa₁₅ is Arg, His, Ser, Ala, Gly, or Lys, and Xaa₁₆ is Leu, Tyr, Phe, or Met.

For example, the CDR-H1 may be a polypeptide having one amino acid sequence selected from the group consisting of amino acid sequences of SEQ ID NOS: 22 to 24, the CDR-H2 may be a polypeptide having an amino acid sequence of SEQ ID NO: 25 or 26, and the CDR-H3 may be a polypeptide having an amino acid sequence of SEQ ID NO: 27 or 28.

Also, the CDR-L1 may be a polypeptide having one amino acid sequence selected from the group consisting of amino acid sequences of SEQ ID NOS: 29 to 33 and 71, CDR-L2 may be a polypeptide having one amino acid sequence selected from the group consisting of amino acid sequences of SEQ ID NOS: 34 to 36, and CDR-L3 may be a polypeptide having one amino acid sequence selected from the group consisting of amino acid sequences of SEQ ID NOS: 13 to 16 or a polypeptide having an amino acid sequence of SEQ ID NO: 37.

According to another embodiment, there is provided an anti-c-Met antibody or antigen binding fragment thereof including: a heavy chain variable region having an amino acid sequence of at least one heavy chain complementarity determining region selected from the group consisting of CDR-H1 having an amino acid sequence of SEQ ID NO: 1, CDR-H2 having an amino acid sequence of SEQ ID NO: 2, and CDR-H3 having an amino acid sequence of SEQ ID NO: 3; and a light chain variable region having an amino acid sequence of at least one light chain complementarity determining region selected from the group consisting of CDR-L1 having an amino acid sequence of SEQ ID NO: 7, CDR-L2 having an amino acid sequence of SEQ ID NO: 8, and CDR-L3 having an amino acid sequence of SEQ ID NO: 9, wherein SEQ ID NOS: 7 to 9 are respectively represented by Formula IV to VI below:

Formula IV
(SEQ ID NO: 7)
Lys-Ser-Ser-Xaa₇-Ser-Leu-Leu-Ala-Xaa₈-Gly-
Asn-Xaa₉-Xaa₁₀-Asn-Tyr-Leu-Ala
Formula V
(SEQ ID NO: 8)
Trp-Xaa₁₁-Ser-Xaa₁₂-Arg-Val-Xaa₁₃
Formula VI
(SEQ ID NO: 9)
Xaa₁₄-Gln-Ser-Tyr-Ser-Xaa₁₅-Pro-Xaa₁₆-Thr

In Formula IV, Xaa₇ is His, Arg, Gln, or Lys, Xaa₈ is Ser or Trp, Xaa₉ is His or Gln, and Xaa₁₀ is Lys or Asn.

In Formula V, Xaa₁₁ is Ala or Gly, Xaa₁₂ is Thr or Lys, and Xaa₁₃ is Ser or Pro.

In Formula VI, Xaa₁₄ is Gly, Ala, or Gln, Xaa₁₅ is Arg, His, Ser, Ala, Gly, or Lys, and Xaa₁₆ is Leu, Tyr, Phe, or Met.

For example, the light chain variable region may have an amino acid sequence of at least one light chain complementarity determining region selected from the group consisting of CDR-L1 having an amino acid sequence of SEQ ID NO: 10 or 71, CDR-L2 having an amino acid sequence of SEQ ID NO: 11, and CDR-L3 having one amino acid sequence selected from the group consisting of amino acid sequences of SEQ ID NOS: 13 to 16.

By way of further illustration, the heavy chain variable region may have an amino acid sequence of SEQ ID NO: 17, and the light chain variable region may have one amino acid sequence selected from the group consisting of amino acid sequences of SEQ ID NOS: 18 to 21 and 72.

The terms “c-Met” or “c-Met protein” may refer to a receptor tyrosine kinase (RTK) that binds to a hepatocyte growth factor (HGF). c-Met can be a c-Met protein from any species, particularly a mammal or primate, for instance, human c-Met (e.g., NP_{—}000236), or monkey c-Met (e.g., Macaca mulatta, NP_{—}001162100), or rodents such as mouse c-Met (e.g., NP_{—}032617.2), rat c-Met (e.g., NP_{—}113705.1), and the like. The c-Met protein may include a polypeptide encoded by the nucleotide sequence identified as GenBank Accession Number NM_{—}000245, a polypeptide having the amino acid sequence identified as GenBank Accession Number NP_{—}000236 or extracellular domains thereof. The receptor tyrosine kinase c-Met participates in various mechanisms, such as cancer development, metastasis, migration of cancer cell, invasion of cancer cell, and angiogenesis.

Animal-derived antibodies produced by immunizing non-immune animals with a desired antigen generally invoke immunogenicity when injected to humans for the purpose of medical treatment, and thus chimeric antibodies have been developed to inhibit such immunogenicity. Chimeric antibodies are prepared by replacing constant regions of animal-derived antibodies that cause an anti-isotype response with constant regions of human antibodies by genetic engineering. Chimeric antibodies are considerably improved in an anti-isotype response compared to animal-derived antibodies, but animal-derived amino acids still have variable regions, so that chimeric antibodies have side effects with respect to a potential anti-idiotype response. Humanized antibodies are developed to reduce such side effects. Humanized antibodies are produced by grafting complementarity determining regions (CDR) which serve an important role in antigen binding in variable regions of chimeric antibodies into a human antibody framework.

The most important thing in CDR grafting to produce humanized antibodies is choosing the optimized human antibodies for accepting CDR of animal-derived antibodies. Antibody database, analysis of a crystal structure, and technology for molecule modeling are used. However, even when the CDRs of animal-derived antibodies are grafted to the most optimized human antibody framework, amino acids positioned in a framework of the animal-derived CDRs affecting antigen binding are present. Therefore, in many cases, antigen binding affinity is not maintained, and thus application of additional antibody engineering technology for recovering the antigen binding affinity is necessary.

The anti-c-Met antibodies may be mouse-derived antibodies, mouse-human chimeric antibodies, or humanized antibodies. The antibodies or antigen-binding fragments thereof may be one isolated from a living body.

The antibody may be a monoclonal antibody.

An intact antibody includes two full-length light chains and two full-length heavy chains, in which each light chain is linked to a heavy chain by disulfide bonds. The antibody has a heavy chain constant region and a light chain constant region. The heavy chain constant region is of a gamma (γ), mu (μ), alpha (α), delta (δ), or epsilon (ε) type, which may be further categorized as gamma 1 (γ1), gamma 2 (γ2), gamma 3 (γ3), gamma 4 (γ4), alpha 1 (α1), or alpha 2 (α2). The light chain constant region is of either a kappa (κ) or lambda (λ) type.

The term “heavy chain” refers to full-length heavy chain, and fragments thereof, including a variable region V_{H} that includes amino acid sequences sufficient to provide specificity to antigens, and three constant regions, C_{H1}, C_{H2}, and C_{H3}, and a hinge. The term “light chain” refers to a full-length light chain and fragments thereof, including a variable region V_{L} that includes amino acid sequences sufficient to provide specificity to antigens, and a constant region C_{L}.

The term “complementarity determining region (CDR)” refers to an amino acid sequence found in a hyper variable region of a heavy chain or a light chain of immunoglobulin. The heavy and light chains may respectively include three CDRs (CDRH1, CDRH2, and CDRH3; and CDRL1, CDRL2, and CDRL3). The CDR may provide contact residues that play an important role in the binding of antibodies to antigens or epitopes. The terms “specifically binding” or “specifically recognized” is well known to one of ordinary skill in the art, and indicates that an antibody and an antigen specifically interact with each other to lead to an immunological activity.

According to an embodiment, the antibody may be an antigen-binding fragment selected from the group consisting of scFv, (scFv)₂, Fab, Fab′, and F(ab′)₂.

The term “antigen-binding fragment” used herein refers to fragments of an intact immunoglobulin including portions of a polypeptide including antigen-binding regions having the ability to specifically bind to the antigen. For example, the antigen-binding fragment may be scFv, (scFv)₂, Fab, Fab′, or F(ab′)₂, but is not limited thereto. Among the antigen-binding fragments, Fab that includes light chain and heavy chain variable regions, a light chain constant region, and a first heavy chain constant region C_{H1}, has one antigen-binding site. The Fab′ fragment is different from the Fab fragment, in that Fab′ includes a hinge region with at least one cysteine residue at the C-terminal of C_{H1}. The F(ab′)₂ antibody is formed through disulfide bridging of the cysteine residues in the hinge region of the Fab′ fragment. Fv is the smallest antibody fragment with only a heavy chain variable region and a light chain variable region. Recombination techniques of generating the Fv fragment are widely known in the art. Two-chain Fv includes a heavy chain variable region and a light chain region which are linked by a non-covalent bond. Single-chain Fv generally includes a heavy chain variable region and a light chain variable region which are linked by a covalent bond via a peptide linker or linked at the C-terminals to have a dimer structure like the two-chain Fv. The antigen-binding fragments may be attainable using protease (for example, the Fab fragment may be obtained by restricted cleavage of a whole antibody with papain, and the F(ab′)₂ fragment may be obtained by cleavage with pepsin), or may be prepared by using a genetic recombination technique.

By way of further example, the anti-c-Met antibody or antibody fragment may include a heavy chain with the amino acid sequence of SEQ ID NO: 62 (wherein the amino acid sequence from 1^{st} to 17^{th} position is a signal peptide) or the amino acid sequence from 18^{th} to 462^{nd} of SEQ ID NO: 62 and a light chain with the amino acid sequence of SEQ ID NO: 68 (wherein the amino acid sequence from 1^{st} to 20^{th} position is a signal peptide) or the amino acid sequence from 21^{st} to 240^{th} position of SEQ ID NO: 68; or a heavy chain with the amino acid sequence of SEQ ID NO: 64 (wherein the amino acid sequence from 1^{st} to 17^{th} position is a signal peptide) or the amino acid sequence from 18^{th} to 461^{st} position of SEQ ID NO: 64 and a light chain with the amino acid sequence of SEQ ID NO: 68 or the amino acid sequence from 21^{st} to 240^{th} position of SEQ ID NO: 68; or a heavy chain with the amino acid sequence of SEQ ID NO: 66 (wherein the amino acid sequence from 1^{st} to 17^{th} position is a signal peptide) or the amino acid sequence from 18^{th} to 460^{th} position of SEQ ID NO: 66 and a light chain with the amino acid sequence of SEQ ID NO: 68 or the amino acid sequence from 21^{st} to 240^{th} position of SEQ ID NO: 68.

Additional examples of anti-c-Met antibodies include those in which the anti-c-Met antibody includes a heavy chain with the amino acid sequence of SEQ ID NO: 62 or the amino acid sequence from 18^{th} to 462^{nd} position of SEQ ID NO: 62 and a light chain with the amino acid sequence of SEQ ID NO: 70 (wherein the amino acid sequence from 1^{st} to 20^{th} position is a signal peptide) or the amino acid sequence from 21^{st} to 240^{th} position of SEQ ID NO: 70; a heavy chain with the amino acid sequence of SEQ ID NO: 64 or the amino acid sequence from 18^{th} to 461^{st} position of SEQ ID NO: 64 and a light chain with the amino acid sequence of SEQ ID NO: 70 or the amino acid sequence from 21^{st} to 240^{th} position of SEQ ID NO: 70; or a heavy chain with the amino acid sequence of SEQ ID NO: 66 or the amino acid sequence from 18^{th} to 460^{th} position of SEQ ID NO: 66 and a light chain with the amino acid sequence of SEQ ID NO: 70 or the amino acid sequence from 21^{st} to 240^{th} position of SEQ ID NO: 70.

In still other examples, the anti-c-Met antibody may include a heavy chain with the amino acid sequence of SEQ ID NO: 62 or the amino acid sequence from 18^{th} to 462^{nd} position of SEQ ID NO: 62 and a light chain with the amino acid sequence of SEQ ID NO: 73; a heavy chain with the amino acid sequence of SEQ ID NO: 64 or the amino acid sequence from 18^{th} to 461^{st} position of SEQ ID NO: 64 and a light chain with the amino acid sequence of SEQ ID NO: 73; or a heavy chain with the amino acid sequence of SEQ ID NO: 66 or the amino acid sequence from 18^{th} to 460^{th} position of SEQ ID NO: 66 and a light chain with the amino acid sequence of SEQ ID NO: 73.

In an embodiment, the anti-c-Met antibody may include a heavy chain with the amino acid sequence from 18^{th} to 460^{th} position of SEQ ID NO: 66 and a light chain with the amino acid sequence from 21^{st} to 240^{th} position of SEQ ID NO: 68; or a heavy chain with the amino acid sequence from 18^{th} to 460^{th} position of SEQ ID NO: 66 and a light chain with the amino acid sequence of SEQ ID NO: 73.

Also provided herein is a polypeptide comprising the amino acid sequence of SEQ ID NO: 68 or 73. The polypeptide with the amino acid sequence of SEQ ID NO: 70 is a light chain including human kappa (κ) constant region, and the polypeptide with the amino acid sequence of SEQ ID NO: 68 is a polypeptide obtained by replacing histidine at position 62 (position 36 according to kabat numbering) of the polypeptide with the amino acid sequence of SEQ ID NO: 70 with tyrosine. The production yield of the antibodies may be increased by the replacement. The polypeptide with the amino acid sequence of SEQ ID NO: 73 is a polypeptide obtained by replacing serine at position 32 (position 27e according to kabat numbering; positioned within CDR-L1) of the polypeptide with the amino acid sequence from 21^{st} to 240^{th} positions of SEQ ID NO: 68 with tryptophan. By such replacement, antibodies and antibody fragments comprising such sequences exhibits increased activities, such as c-Met biding affinity, c-Met degradation activity, Akt phosphorylation activity, and the like.

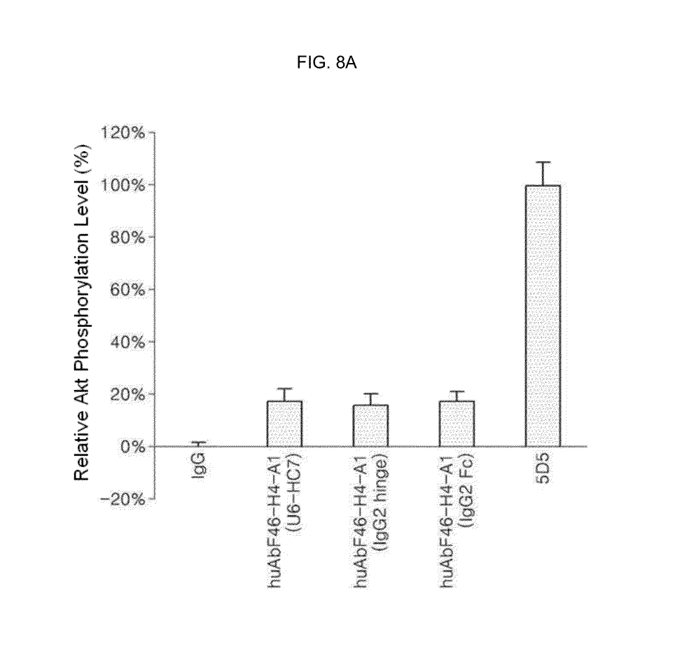

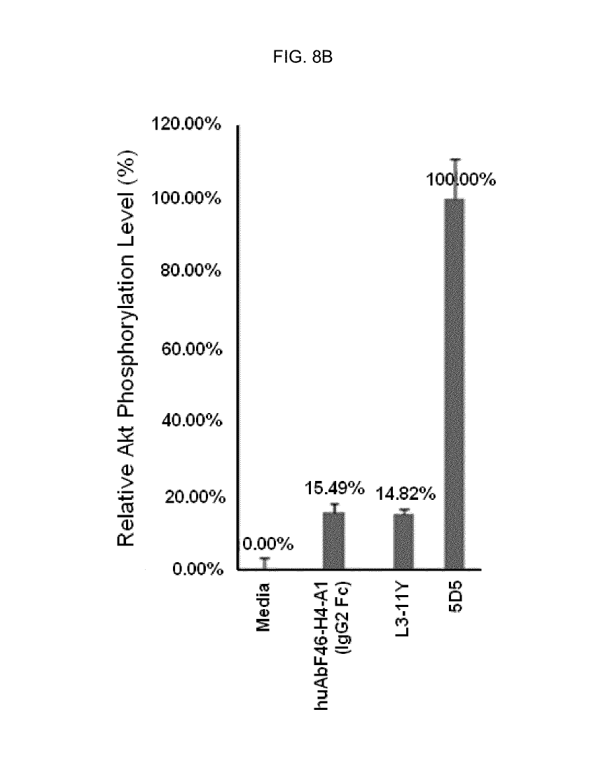

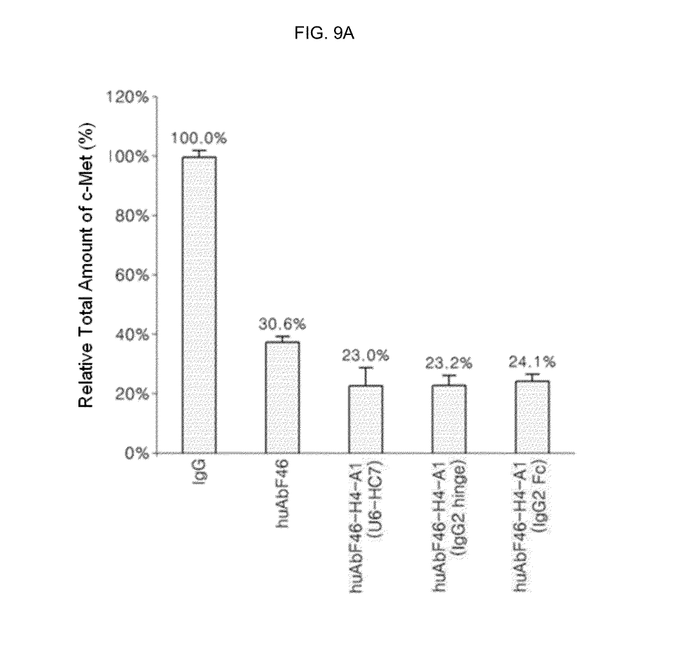

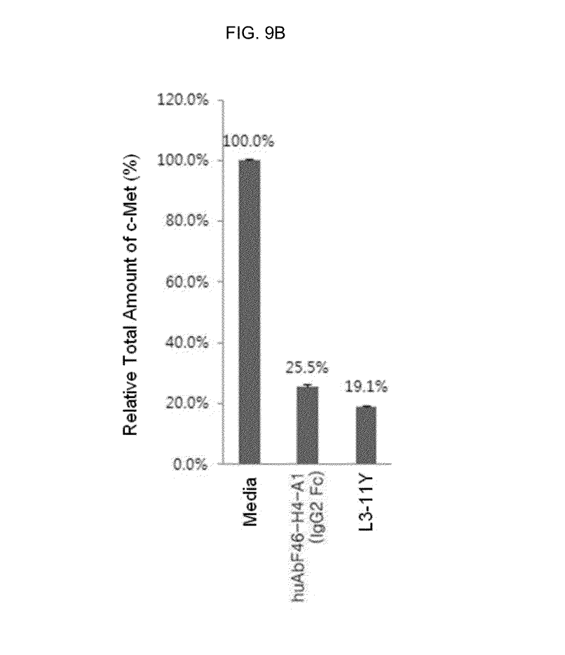

Another embodiment provides a polypeptide having the amino acid sequence of SEQ ID NO: 71, which is useful as a light chain complementarity determining region (CDR-L1). Another embodiment provides a anti-c-Met antibody or an antigen-binding fragment thereof including a light chain complementarity determining region having the amino acid sequence of SEQ ID NO: 71, a light chain variable region having the amino acid sequence of SEQ ID NO: 72, or a light chain having the amino acid sequence of SEQ ID NO: 73, optionally in combination with a heavy chain variable region or heavy chain as described herein, or other heavy chain that provides an anti-c-Met antibody or antibody fragment. The antibody or the antigen-binding fragment thereof exhibits increased c-Met degradation activity and Akt phosphorylation activity, as shown in FIGS. 8B and 9B.

According to another embodiment, there is provided a pharmaceutical composition including the anti-c-Met antibody or the antigen-binding fragment as an active ingredient. The pharmaceutical composition can be used for preventing or treating a cancer or for preventing or inhibition of metastasis of a cancer, and may include a pharmaceutically effective amount of the anti-c-Met antibody or the antigen-binding fragment; and a pharmaceutically acceptable carrier, a diluent, or an excipient.

The cancer may be any cancer associated with c-Met activity or overexpression (high level) of c-Met. The cancer may be any selected from the group consisting of squamous cell carcinoma, small-cell lung cancer, non-small-cell lung cancer, adenocarcinoma of the lung, squamous cell carcinoma of the lung, peritoneal carcinoma, skin cancer, skin or intraocular melanoma, colorectal cancer, cancer near the anus, esophagus cancer, small intestinal tumor, endocrine gland cancer, parathyroid cancer, adrenal cancer, soft-tissue sarcoma, urethral cancer, chronic or acute leukemia, lymphocytic lymphoma, hepatoma, gastrointestinal cancer (gastric cancer), pancreatic cancer, glioblastoma, cervical cancer, ovarian cancer, liver cancer, bladder cancer, hepatocellular adenoma, breast cancer, colon cancer, large intestine cancer, endometrial or uterine carcinoma, salivary gland tumor, kidney cancer, prostate cancer, vulvar cancer, thyroid cancer, and head and neck cancers. The cancer may include a metastatic cancer as well as a primary cancer.

The pharmaceutical composition may include a pharmaceutically acceptable carrier, a diluent, and/or excipient. The pharmaceutically acceptable carriers included in the composition may include commonly used lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and mineral oil, but is not limited thereto. The pharmaceutical composition may further include a lubricant, a wetting agent, a sweetener, a flavor enhancer, an emulsifying agent, a suspension agent, and a preservative.

The pharmaceutical composition may be administered orally or parenterally. The parenteral administration may include intravenous injection, subcutaneous injection, muscular injection, intraperitoneal injection, endothelial administration, local administration, intranasal administration, intrapulmonary administration, and rectal administration. Since oral administration leads to digestions of protein or peptide, an active ingredient must be coated or formulated in a pharmaceutical composition, digestion of which is prevented. In addition, the pharmaceutical composition may be administered by using any device capable of moving an active material toward a target cell.

A suitable dosage of the pharmaceutical composition may depend on many factors, such as formulation methods, administration methods, ages, body weight, gender, and pathologic conditions of patients, diets, administration time, administration route, excretion speed, and reaction sensitivity. The desirable dose of the pharmaceutical composition may be in the range of about 0.001 to 100 mg/kg for an adult. The term “pharmaceutically effective amount” or “therapeutically effective amount” used herein refers to an amount used in preventing or treating cancer and/or angiogenesis-related diseases.

The pharmaceutical composition may be formulated, with a pharmaceutically acceptable carrier and/or an additive, into a unit or a multiple dosage form by a well-known method in the art. In this regard, the formulation may be a solution in oil or an aqueous medium, a suspension, a syrup, an emulsifying solution, an extract, powder, granules, a tablet, or a capsule, and may further include a dispersing or a stabilizing agent. In addition, the pharmaceutical composition may be administered as an individual drug, or together with other drugs, and may be administered sequentially or simultaneously with pre-existing drugs. The pharmaceutical composition includes the antibody or the antigen-binding fragments thereof, and thus may be formulated as an immunoliposome. The liposome containing the antibody may be prepared using a well-known method in the art. The immunoliposome is a lipid composition including phosphatidylcholine, cholesterol, and polyethyleneglycol-derived phosphatidylethanolamine, and may be prepared by a reverse phase evaporation method. For example, Fab′ fragments may be adhered to the liposome through a disulfide exchange reaction. A chemical drug, such as doxorubicin, may also be included in the liposome.

According to an embodiment, the antibody or antibody fragment may act as an antagonist against the c-Met protein.

The term “antagonist” is understood to include all molecules that partially or entirely block, inhibit, and/or neutralize at least one biological activity of their targets (e.g., c-Met). For example, the term “antagonist antibody” refers to an antibody that inhibits or decreases the biological activity of an antigen to which the antibody binds (e.g., c-Met). An antagonist may decrease receptor phosphorylation due to binding receptors to ligands, promote degredation, or may incapacitate or destroy cells that are activated by the ligands. Also, an antagonist may completely block interaction between a receptor and a ligand, or may practically decrease the interaction due to tertiary structure change or down regulation of the receptor.

According to another embodiment, there is provided a method of preventing and/or treating a cancer, the method including administering the anti-c-Met antibody or the antigen-binding fragment to a subject in need of preventing and/or treating a cancer. In another embodiment, there is provided a method of preventing and/or inhibiting metastasis of a cancer, the method including administering the anti-c-Met antibody or the antigen-binding fragment to a subject in need of preventing and/or inhibiting metastasis of a cancer. The antibody or antibody fragment may be administered in a pharmaceutically effective amount, and may be administered as a pharmaceutical composition formulated with a pharmaceutically acceptable carrier, a diluent, or excipient, as described herein. The method may further include identifying a subject in need of preventing and/or treating a cancer or preventing and/or inhibiting metastasis of a cancer, prior to the administering step. The cancer is described as above.

According to another embodiment, there is provided the anti-c-Met antibody or the antigen-binding fragment for use in preventing and/or treating a cancer, or preparing a medicament for preventing and/or treating a cancer.

The subject to which the active ingredient(s) or the pharmaceutical composition may be administered includes an animal, such as a mammal. For example, the animal may be a human, dog, cat, or mouse.

According to another embodiment of the present invention, there is provided a nucleic acid encoding an antibody or antigen binding fragment thereof as described herein, as well as a nucleic acid encoding any of the foregoing polypeptides or amino acid sequences. The nucleic acid encoding the antibody or antigen binding fragment thereof may be, for example, DNA or RNA and may optionally be incorporated in a vector, such as an expression vector.

According to another embodiment of the present invention, there is provided a cell comprising a nucleic acid encoding an antibody or antigen binding fragment thereof as described herein, as well as a nucleic acid encoding any of the foregoing polypeptides or amino acid sequences.

According to another embodiment of the present invention, there is provided a method of preparing an antibody or antigen binding fragment thereof as described herein, the method comprising expressing a nucleic acid encoding the antibody or antigen binding fragment thereof in a cell.

One or more embodiments of the present invention will now be described in further detail with reference to the following Examples. However, these examples are for the illustrative purposes only and are not intended to limit the scope of the invention.

### Example 1

### Preparation of Mouse Antibody AbF46 Against c-Met

(1) Immunization of Mice

To obtain immunized mice necessary for developing hybridoma cell lines, 100 ug of human c-Met/Fc fusion protein (R&D Systems) and a complete Freund's adjuvant in the same amount were mixed, and the mixture was administered via an intraperitoneal injection to each of five 4 to 6-week-old BALB/c mice (Japan SLC, Inc.). After two weeks, the antigen (half the previously injected amount) was mixed with an incomplete Freund's adjuvant using the same method as described above, and the mixture was administered to each mouse via an intraperitoneal injection. After one week, final boosting was performed, and blood was collected from the tail of each mouse after three days to obtain serum. Then, serum was diluted at 1/1000 with PBS, and an enzyme-linked immunosorbent assay (ELISA) was performed to analyze whether the titer of the antibody recognizing c-Met increased. Afterwards, mice in which a sufficient amount of the antibody was obtained were selected, and a cell fusion process was performed on the selected mice.

(2) Cell Fusion and Preparation of Hybridoma Cells

Three days before a cell fusion experiment, a mixture of 50 ug of PBS and human c-Met/Fc fusion protein was administered via an intraperitoneal injection to each mouse (BALB/c mice; Japan SLC, Inc.). Each immunized mouse was anesthetized, and its spleen located on the left side of the body was then extracted and ground with a mesh to isolate cells, which were mixed with a culture medium (DMEM, GIBCO, Invitrogen) to prepare a spleen cell suspension. The suspension was centrifuged to collect a cell layer. The obtained 1×10⁸ spleen cells were mixed with 1×10⁸ myeloma cells (Sp2/0), and the mixture was centrifuged to precipitate the cells. The precipitate was slowly dispersed, treated with 1 ml of 45% (w/v) polyethylene glycol (PEG) in DMEM, and maintained at 37° C. for one minute before adding 1 ml of DMEM. After introducing additional 10 ml of DMEM for 1 minute, the resultant was maintained in a water bath at 37° C. for 5 minutes. The total amount thereof was made to reach 50 ml, and the resultant was centrifuged. The resulting cell precipitate was re-suspended in an isolation medium (HAT medium) at a concentration of 1×10⁵ cells/ml to 2×10⁵ cells/ml. Then, the resultant was distributed to a 96-well plate (0.1 ml per well), which was incubated in a carbon dioxide incubator at 37° C. to prepare the hybridoma cells.

(3) Selection of Hybridoma Cells that Produce Monoclonal Antibodies Against c-Met Protein

To select the hybridoma cells that specifically bind to c-Met from the hybridoma cells prepared in operation (2) described above, ELISA was performed to screen for the cells that produced antibodies active against human c-Met/Fc fusion protein and human Fc protein.

50 ul (2 ug/ml) of human c-Met/Fc fusion protein was coated on each well of a microtiter plate, and unreacted antigens were removed by washing. To exclude antibodies binding to Fc, but not to c-Met, the human Fc protein was coated on each well of a different microtiter plate using the same method as above. Then, 50 ul of a hybridoma cell suspension was added to each well of the microtiter plates to react for 1 hour. Then, the microwell plates were washed with a phosphate buffer-tween 20 (TBST) solution to remove unreacted culture medium. Goat anti-mouse IgG-horseradish peroxidase (IgG-HRP) was added thereto, and a reaction was allowed to occur at room temperature for 1 hour, and washing was performed with the TBST solution. Subsequently, a substrate solution (OPD) of peroxidase was added to each well, and the reaction degree was evaluated by measuring the absorption at 450 nm using an ELISA reader. Through this method, hybridoma cell lines that produce antibodies highly specific to the human c-Met protein and not to the human Fc protein were repeatedly selected. A limiting dilution was performed on the obtained hybridoma cell lines to obtain a single clone of hybridoma cell lines producing monoclonal antibodies. The selected hybridoma cell line producing the monoclonal antibody was registered in the Korean Cell Line Bank with accession number KCLRF-BP-00220 (deposited Oct. 6, 2009 with the Korean Cell Line Research Foundation, Cancer Research Institute, Seoul National University College of Medicine, 28 Yongon-dong, Chongno-Gu, Seoul, 110-744, Korea).

(4) Production and Purification of the Monoclonal Antibody

The hybridoma cells obtained in operation (3) described above were cultured in a serum free medium to produce monoclonal antibodies and the monoclonal antibodies were purified.

First, the hybridoma cells cultured in 50 ml of culture medium (DMEM) with 10% (w/v) FBS were centrifuged to obtain a cell precipitate, which was washed with 20 ml of PBS more than twice to remove the FBS. Then, 50 ml of DMEM was introduced to re-suspend the cell precipitate, and the resultant was incubated in a carbon dioxide incubator at 37° C. for 3 days. After centrifugation to remove antibody-producing cells, cell culture including antibodies was isolated and stored at 4° C., or used directly. Antibodies were purified from 50 to 300 ml of the culture using a AKTA purification device (GE Health) equipped with an affinity column (protein G agarose column; Pharmacia, USA), and the purified antibodies were stored by replacing the supernatant with PBS using a filter for protein aggregation (Amicon).

### Example 2

### Preparation of Chimeric Antibody chAbF46 Against c-Met

Generally, when a mouse antibody is injected into a human for medical purposes, immunogenicity may often occur. Thus, to reduce the immunogenicity, a chimeric antibody chAbF46, in which the constant region is substituted with the amino acid sequence of a human IgG1 antibody, was prepared from the mouse antibody AbF46 prepared in Example 1.

Genes were synthesized such that nucleic acid sequence corresponding to a heavy chain was EcoRI-signal sequence-VH-NheI-CH-TGA-XhoI (SEQ ID NO: 38) and nucleic acid sequence corresponding to a light chain was EcoRI-signal sequence-VL-BsiWI-CL-TGA-XhoI (SEQ ID NO: 39). Then, vectors for expression of a chimeric antibody was constructed by cloning a DNA fragment (SEQ ID NO: 38) having the nucleic acid sequence corresponding to the heavy chain in a pOptiVEC™-TOPO TA Cloning Kit included in an OptiCHO™ Antibody Express Kit (Cat No. 12762-019) manufactured by Invitrogen and a DNA fragment (SEQ ID NO: 39) having the nucleic acid sequence corresponding to the light chain in a pcDNA™3.3-TOPO TA Cloning Kit (Cat No. 8300-01) by using restriction enzymes, EcoRI(NEB, R0101S) and XhoI(NEB, R0146S), respectively.

The constructed vectors were amplified using a Qiagen Maxiprep kit (Cat No. 12662), and vectors including the heavy chain and vectors including the light chain were added to 293T cells (2.5×10⁷) at a ratio of about 4:1 (about 80 ug:20 ug) with 360 ul of 2 M CaCl₂ and were transfected. Next, the mixture was cultured in a DMEM medium with 10% (w/v) FBS at 37° C. in 5% (v/v) CO₂ conditions for 5 hours, and then cultured in a DMEM medium without FBS at 37° C. in 5% (v/v) CO₂ conditions for 48 hours.

The cultured cells were centrifuged, and 100 ml of each supernatant was purified using AKTA Prime (GE healthcare). Protein A column (GE healthcare, 17-0405-03) was placed in the AKTA Prime, and the cultured solution was flowed at a flow rate of 5 ml/min and was eluted with IgG elution buffer (Thermo Scientific, 21004). The buffer was replaced with a PBS buffer, and thus a final chimeric antibody (hereinafter, chAbF46) was purified.

### Example 3

### Preparation of Humanized Antibody huAbF46 from Chimeric Antibody chAbF46

(1) Heavy Chain Humanization

For the H1-heavy chain and the H3-heavy chain, the human germline gene most homologous to a VH gene of mouse antibody AbF46 was identified using NCBI Ig Blast. VH3-71 was confirmed to have 83% homology at an amino acid level. CDR-H1, CDR-H2, and CDR-H3 of mouse antibody AbF46 were numbered using Kabat numbering and a CDR portion of mouse antibody AbF46 was introduced in a framework of VH3-71. Amino acids of No. 30 (S→T), No. 48 (V→L), No. 73 (D→N), and No. 78 (T→L) were back-mutated to the amino acid sequence of the original mouse AbF46 antibody, wherein the number of the amino acid is numbered according to Kabat numbering, and thus, the number is common to the VH3-71 and mouse AbF46 antibody. Then, in the H1-heavy chain, the amino acids of No. 83 (R→K) and No. 84 (A→T) were additionally mutated, thereby completing construction of H1-heavy chain (SEQ ID NO: 40) and H3-heavy chain (SEQ ID NO: 41).

For the H4-heavy chain, a framework sequence of a human antibody was obtained, and the VH3 subtype (known to have a sequence similar to the mouse framework sequence of the AbF46 antibody and to be stable) was used to introduce CDR-H1, CDR-H2, and CDR-H3 of mouse antibody AbF46 defined using Kabat numbering. Accordingly, the H4-heavy chain (SEQ ID NO: 42) was constructed.

(2) Light Chain Humanization

For the H1-light chain (SEQ ID NO: 43) and the H2-light chain (SEQ ID NO: 44), the human germline gene most homologous to the VL gene of mouse antibody AbF46 was identified using NCBI Ig Blast. VK4-1 was confirmed to have 75% of homology at the amino acid level. CDR-L1, CDR-L2, and CDR-L3 of mouse antibody AbF46 were defined using Kabat numbering and a CDR portion of mouse antibody AbF46 was introduced into a framework of VK4-1. In the H1-light chain, 3 amino acids of No. 36 (Y→H), No. 46 (L→M), and No. 49 (Y→I) were back-mutated. In the H2-light chain, only one amino acid of No. 49 (Y→I) was back-mutated.

For the H3-light chain (SEQ ID NO: 45), the human germline gene most homologous to the VL gene of mouse antibody AbF46 was identified using NCBI Ig Blast. As a result, VK2-40 in addition to VK4-1 (mentioned above) was chosen. Mouse antibodies AbF46 VL and VK2-40 were confirmed to have 61% homology at an amino acid level. CDR-L1, CDR-L2, and CDR-L3 of mouse antibody AbF46 were defined using Kabat numbering and a CDR portion of the mouse antibody AbF46 was introduced into a framework of VK4-1. In the H3-light chain, 3 amino acids of No. 36 (Y→H), No. 46 (L→M), and No. 49 (Y→I) were back-mutated.

For the H4-light chain (SEQ ID NO: 46), a framework sequence of a human antibody was obtained, and the VK1 subtype (conventionally known to be stable) was used to introduce CDR-L1, CDR-L2, and CDR-L3 of mouse antibody AbF46 defined using Kabat numbering. In the H4-light chain, 3 amino acids of No. 36 (Y→H), No. 46 (L→M), and No. 49 (Y→I) were additionally back-mutated.

Then, vectors for expression of the humanized antibody were constructed by cloning DNA fragments (H1-heavy; SEQ ID NO: 47, H3-heavy; SEQ ID NO: 48, and H4-heavy; SEQ ID NO: 49) having the nucleic acid sequence corresponding to the heavy chain in a pOptiVEC™-TOPO TA Cloning Kit included in an OptiCHO™ Antibody Express Kit (Cat No. 12762-019) manufactured by Invitrogen and DNA fragments (H1-light; SEQ ID NO: 50, H2-light; SEQ ID NO: 51, H3-light; SEQ ID NO: 52, and H4-light; SEQ ID NO: 53) having the nucleic acid sequence corresponding to the light chain in a pcDNA™3.3-TOPO TA Cloning Kit (Cat No. 8300-01) by using restriction enzymes, EcoRI(NEB, R0101S) and XhoI(NEB, R0146S), respectively.

The constructed vectors were amplified using a Qiagen Maxiprep kit (Cat No. 12662), and vectors including the heavy chain and vectors including the light chain were added to 293T cells (2.5×10⁷) at a ratio of about 4:1 (about 80 ug: 20 ug) with 360 ul of 2 M CaCl₂ and were transfected. Next, the mixture was cultured in a DMEM medium added with 10% (w/v) FBS at 37° C. in 5% (v/v) CO₂ conditions for 5 hours, and then cultured in a DMEM medium without FBS at 37° C. in 5% (v/v) CO₂ conditions for 48 hours.

The cultured cells were centrifuged, and 100 ml of each supernatant was purified using AKTA Prime (GE healthcare). Protein A column (GE healthcare, 17-0405-03) was placed in the AKTA Prime, and the cultured solution was flowed at a flow rate of 5 ml/min and was eluted with IgG elution buffer (Thermo Scientific, 21004). The buffer was exchanged with a PBS buffer, and thus a final humanized antibody (hereinafter, huAbF46) was purified. The combination of the H4-heavy chain and the H4-light chain of humanized huAbF46 were used hereinafter.

### Example 4

### Preparation of scFv Library of huAbF46 Antibody

Genes for preparing scFv of huAbF46 antibody were designed by using the heavy chain variable region and light chain variable region of huAbF46 antibody. Each of the heavy chain variable region and light chain variable region was designed to have a ‘VH-linker-VL’ form, in which the linker was designed to have an amino acid sequence of ‘GLGGLGGGGSGGGGSGGSSGVGS’ (SEQ ID NO: 54). A polynucleotide (SEQ ID NO: 55) encoding scFv of huAbF46 antibody designed as described above was synthesized (Bioneer, Inc.), and a vector for expressing the polynucleotide was represented as SEQ ID NO: 56.

Then, resultants expressed by the vector were analyzed, and c-Met specific binding was identified.

### Example 5

### Preparation of Library Gene for Affinity Maturation

(1) Selection of Target CDR and Preparation of Primer

For affinity maturation of huAbF46 antibody, 6 complementarity determining regions (CDRs) were defined by ‘Kabat numbering’ from the prepared mouse antibody AbF46. CDRs are shown in Table 1.

TABLE 1

| CDR | amino acid sequence |
|---|---|
| CDR-H1 | DYYMS (SEQ ID NO: 1) |
| CDR-H2 | FIRNKANGYTTEYSASVKG (SEQ ID NO: 2) |
| CDR-H3 | DNWFAY (SEQ ID NO: 3) |
| CDR-L1 | KSSQSLLASGNQNNYLA (SEQ ID NO: 10) |
| CDR-L2 | WASTRVS (SEQ ID NO: 11) |
| CDR-L3 | QQSYSAPLT (SEQ ID NO: 12) |

Primers were prepared as follows in order to randomly introduce sequences of CDRs of antibody. According to existing methods of randomly introducing sequences, N codon was used such that bases could be introduced into sites to be mutated at the same rate (25% A, 25% G, 25% C, and 25% T). However, according to the current embodiment, in order to randomly introduce bases into the CDRs of the huAbF46 antibody, 85% of the first and second nucleotides were preserved among three wild-type nucleotides coding amino acids of each CDR, and 5% of each of the other three bases was introduced. In addition, the primer was designed such that the three bases could be introduced into the third nucleotide (33% G, 33% C, and 33% T).

(2) Preparation of huAbF46 Antibody Library and Identification of Binding Affinity to c-Met

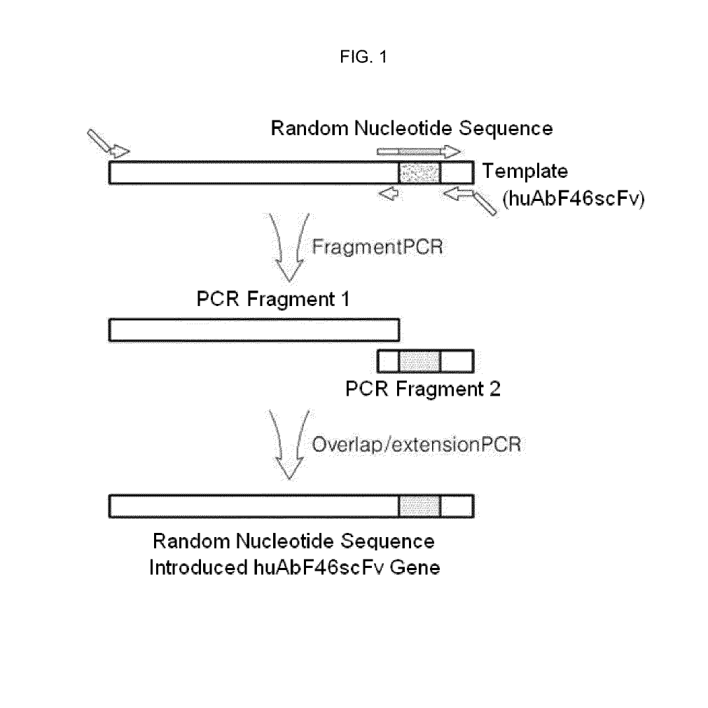

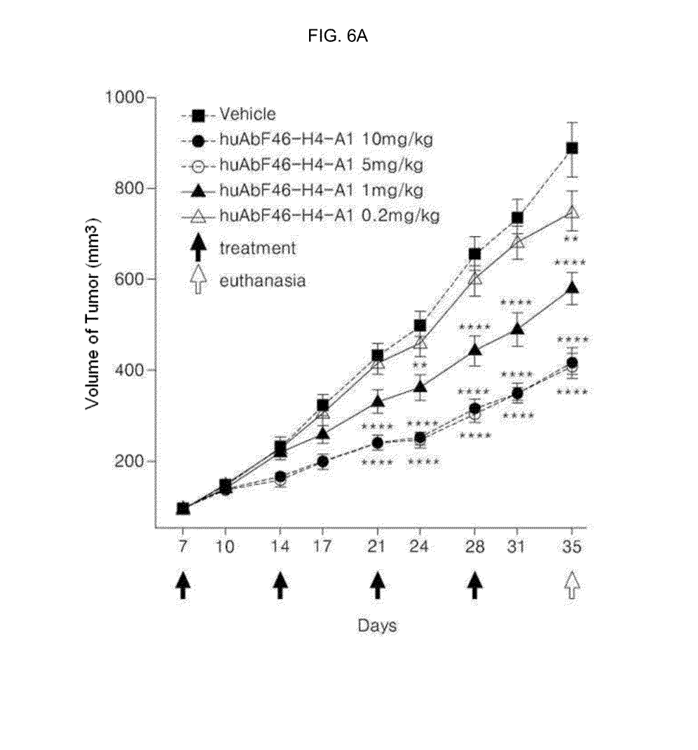

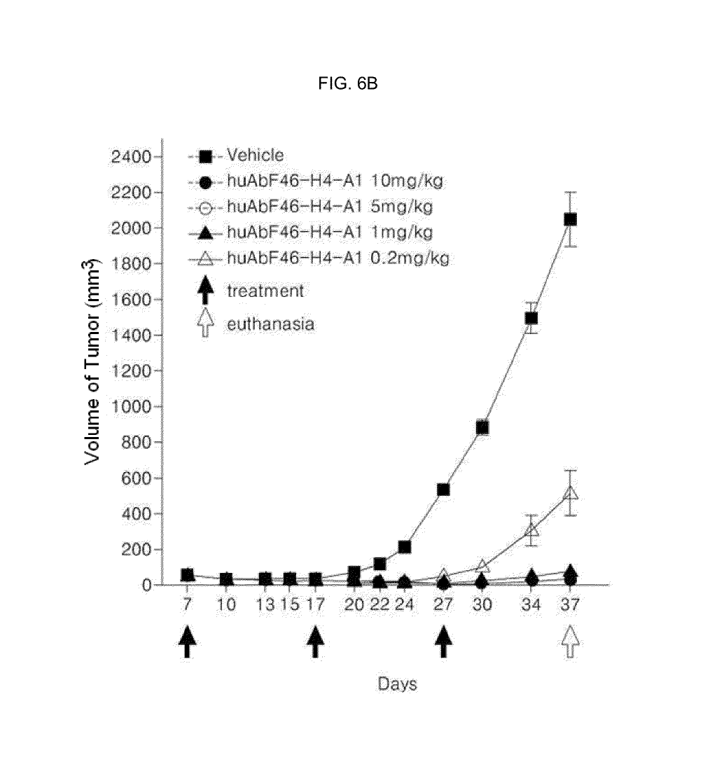

The construction of an antibody gene library by randomly introducing sequences into CDRs was performed using the primer prepared in operation (1) described above. A polynucleotide including nucleic acid sequence encoding scFv of the huAbF46 antibody was used as a template. Two PCR fragments were prepared as shown in FIGS. 1 and 6 libraries, respectively targeting the 6 CDRs were constructed by using an overlap extension PCR.

The binding affinities of the wild-type antibody (huAb46) and each antibody derived from the libraries to c-Met were identified. While the binding affinities of most antibodies derived from the libraries to c-Met were lower than that of the wild-type antibody, mutants in which the binding affinity to c-Met was not reduced were identified.

### Example 6

### Selection of Antibody with Improved Affinity from the Libraries

If the binding affinity of an antibody derived from the libraries to c-Met was improved, the scFv gene sequence from that individual clone was analyzed. The obtained CDR sequences, shown in Table 2 below, were transformed into IgG. Among the clones listed below, 4 types of antibodies produced from L3-1, L3-2, L3-3, and L3-5 were selected and subsequent experiments were performed using these antibodies.

TABLE 2

| Name of clone | Library | CDR sequence |
|---|---|---|
| H11-4 | CDR-H1 | PEYYMS (SEQ ID NO: 22) |
| YC151 | CDR-H1 | PDYYMS (SEQ ID NO: 23) |
| YC193 | CDR-H1 | SDYYMS (SEQ ID NO: 24) |
| YC244 | CDR-H2 | RNNANGNT (SEQ ID NO: 25) |
| YC321 | CDR-H2 | RNKVNGYT (SEQ ID NO: 26) |
| YC354 | CDR-H3 | DNWLSY (SEQ ID NO: 27) |
| YC374 | CDR-H3 | DNWLTY (SEQ ID NO: 28) |
| L1-1 | CDR-L1 | KSSHSLLASGNQNNYLA |
| | | (SEQ ID NO: 29) |
| L1-3 | CDR-L1 | KSSRSLLSSGNHKNYLA |
| | | (SEQ ID NO: 30) |
| L1-4 | CDR-L1 | KSSKSLLASGNQNNYLA |
| | | (SEQ ID NO: 31) |
| L1-12 | CDR-L1 | KSSRSLLASGNQNNYLA |
| | | (SEQ ID NO: 32) |
| L1-22 | CDR-L1 | KSSHSLLASGNQNNYLA |
| | | (SEQ ID NO: 33) |
| L2-9 | CDR-L2 | WASKRVS (SEQ ID NO: 34) |
| L2-12 | CDR-L2 | WGSTRVS (SEQ ID NO: 35) |
| L2-16 | CDR-L2 | WGSTRVP (SEQ ID NO: 36) |
| L3-1 | CDR-L3 | QQSYSRPYT (SEQ ID NO: 13) |
| L3-2 | CDR-L3 | GQSYSRPLT (SEQ ID NO: 14) |
| L3-3 | CDR-L3 | AQSYSHPFS (SEQ ID NO: 15) |
| L3-5 | CDR-L3 | QQSYSRPFT (SEQ ID NO: 16) |
| L3-32 | CDR-L3 | QQSYSKPFT (SEQ ID NO: 37) |

### Example 7

### Transformation of Selected Antibodies to IgG

A polynucleotide encoding the heavy chain of the selected 4 types of antibodies consisted of ‘EcoRI-signal sequence-VH-NheI-CH-XhoI’ (SEQ ID NO: 38). The amino acids of the heavy chain were not modified after affinity was matured, so the heavy chain of the huAbF46 antibody was used. The hinge region was replaced with a U6-HC7 hinge region (SEQ ID NO: 57), not with the hinge region of human IgG1. A gene of the light chain was designed to have ‘EcoRI-signal sequence-VL-BsiWI-CL-XhoI’, and polynucleotides (SEQ ID NOS: 58 to 61) encoding light chain variable regions of the selected 4 types of antibodies were synthesized by Bioneer, Inc. Then, vectors for expression of the antibodies were constructed by cloning a DNA fragment (SEQ ID NO: 38) having the nucleic acid sequence corresponding to the heavy chain in a pOptiVEC™-TOPO TA Cloning Kit included in an OptiCHO™ Antibody Express Kit (Cat No. 12762-019) manufactured by Invitrogen and DNA fragments (a DNA fragment including L3-1-derived CDR-L3 (SEQ ID NO: 58), a DNA fragment including L3-2-derived CDR-L3 (SEQ ID NO: 39), a DNA fragment including L3-3-derived CDR-L3 (SEQ ID NO: 60), and a DNA fragment including L3-5-derived CDR-L3 (SEQ ID NO: 61)) corresponding to the light chain in a pcDNA™3.3-TOPO TA Cloning Kit (Cat No. 8300-01) by using a restriction enzyme, EcoRI(NEB, R0101S) and XhoI(NEB, R0146S), respectively.

The constructed vectors were amplified using a Qiagen Maxiprep kit (Cat No. 12662), and vectors including the heavy chain and vectors including the light chain were added to 293T cells (2.5×10⁷) at a ratio of about 4:1 (about 80 ug: 20 ug) with 360 ul of 2 M CaCl₂ and were transfected. Next, the mixture was cultured in a DMEM medium with 10% (w/v) FBS at 37° C. in 5% (v/v) CO₂ conditions for 5 hours, and then cultured in a DMEM medium without FBS at 37° C. in 5% (v/v) CO₂ conditions for 48 hours.

The cultured cells were centrifuged, and 100 ml of each supernatant was purified using AKTA Prime (GE healthcare). Protein A column (GE healthcare, 17-0405-03) was placed in the AKTA Prime, and the cultured solution was flowed at a flow rate of 5 ml/min and was eluted with IgG elution buffer (Thermo Scientific, 21004). The buffer was exchanged with a PBS buffer, and thus 4 types of antibodies having improved affinities (hereinafter, huAbF46-H4-A1, huAbF46-H4-A2, huAbF46-H4-A3, and huAbF46-H4-A5) were purified.

### Example 8

### Analysis of Binding Affinity of Selected Antibodies

Affinities of the 4 types of antibodies against c-Met antigen prepared in Example 7 were measured by using a Biacore (GE healthcare). About 80 to 110 RU of each antibody was immobilized on a CM5 chip, 9 different concentrations ranging from 0.39 nM to 100 nM of human c-Met protein, as an antigen, were injected at a rate of 30 ul/min to obtain kₒₙ values and k_{off} values as shown in Table 3. Then, K_{D} values were calculated based thereon. A binding affinity of huAbF46 to c-Met antigen was about 2.19 nM, and binding affinities of the four types of antibodies having improved affinities were in a range of 0.06 nM to 0.50 nM (Table 3). This indicates that affinities of the antibodies, which were improved in the form of scFv, were further improved by about 5 times to about 37 times after being transformed to IgG.

TABLE 3

| Antibody | kₒₙ (1/Ms) | k_{off} (1/s) | K_{D} (nM) |
|---|---|---|---|
| huAbF46 | 3.29 × 10⁵ | 7.23 × 10⁻⁴ | 2.19 |
| huAbF46-H4-A1 | 7.39 × 10⁵ | 4.53 × 10⁻⁵ | 0.06 |
| huAbF46-H4-A2 | 5.02 × 10⁵ | 2.53 × 10⁻⁴ | 0.50 |
| huAbF46-H4-A3 | 4.19 × 10⁵ | 1.43 × 10⁻⁴ | 0.34 |
| huAbF46-H4-A5 | 5.72 × 10⁵ | 2.40 × 10⁻⁴ | 0.42 |

### Example 9

### Analysis of In Vitro Biological Activity of Selected Antibodies Having Improved Affinities

(1) BrdU Assay

A BrdU assay was performed using the antibodies having improved affinities in order to evaluate safety of the antibodies. NCI-H441 (ATCC Cat. # HTB-174), human lung cancer cells, were suspended in a RPMI 1640 medium (Gibco) (2×10⁵ cell/ml) to prepare a suspension, and about 100 ul of the suspension was introduced to each well of a 96-well tissue culture plate (Corning, Lowell, Mass.). The suspension was incubated at 37° C. in 5% (v/v) CO₂ conditions for 24 hours, and then the medium was completely removed and replaced with a RPMI 1640 diluted with the antibody. After incubating the suspension at 37° C. in 5% (v/v) CO₂ conditions for 21 hours, 5-bromo-2′-deoxyuridine (BrdU) was added and the BrdU assay (Roche, Indianapolis, Ind.) was performed after a further 3 hours of incubation. After denaturing/fixing cells on the plate, an anti-BrdU antibody was added thereto and a substrate was added after an hour to measure a color reaction using an ELISA spectraMax reader (Molecular Devices, Sunnyvale, Calif.) at 370 nm. Media was used as a negative control, and an antibody 5D5 (ATCC Cat. # HB11895 separated from hybridoma cells and purified) well known as an agonist was used as a positive control.

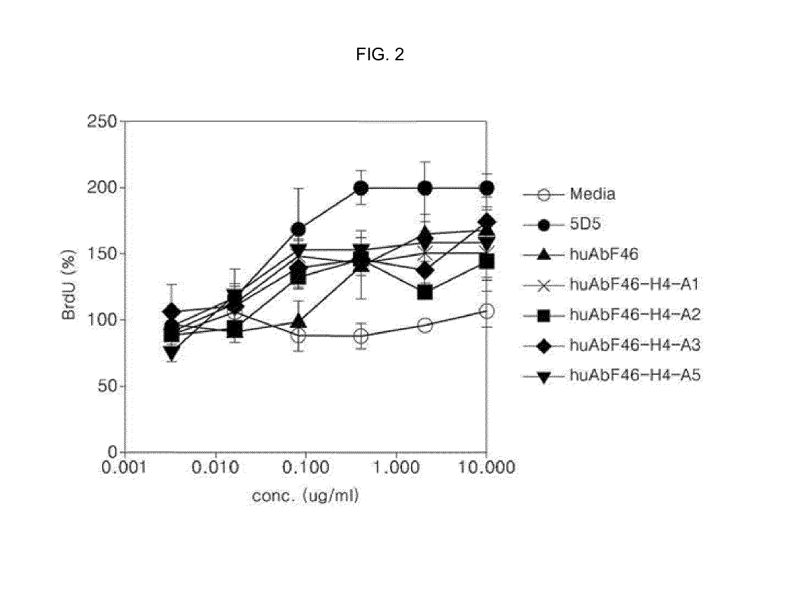

As a result, referring to FIG. 2, among the 4 types of antibodies having improved affinities, agonism side effects of 4 types were reduced. Thus, it was identified that safeties thereof were respectively improved by 25% (huAbF46-H4-A1), 28% (huAbF46-H4-A2), 13% (huAbF46-H4-A3), and 21% (huAbF46-H4-A5) at a concentration of 10 ug/ml.

(2) In Vitro Cell Proliferation Analysis

In order to identify anti-cancer effects of the 4 types of antibodies having improved affinities, as prepared in Example 5, in vitro cell proliferation analysis was performed using MKN45 gastric cancer cells on which c-Met is expressed (Japanese Cancer Research Bank, JCRB, Tokyo, Japan).

1×10⁴ MKN45 cells suspended in 50 ul of 5% (w/v) FBS/DMEM culture medium were introduced to each well of a 96-well plate. Then, the cells were either not treated or were treated with 50 ul of the 4 types of antibodies at a concentration of 0.008, 0.04, 0.2, or 1 ug/ml. After incubating for 72 hours, the number of cells was quantified by using a CellTiter-Glo Luminescent Cell Viability Assay Kit (Promega, G7570) with a leuminometer (PerkinElmer, 2104 Multilabel reader).

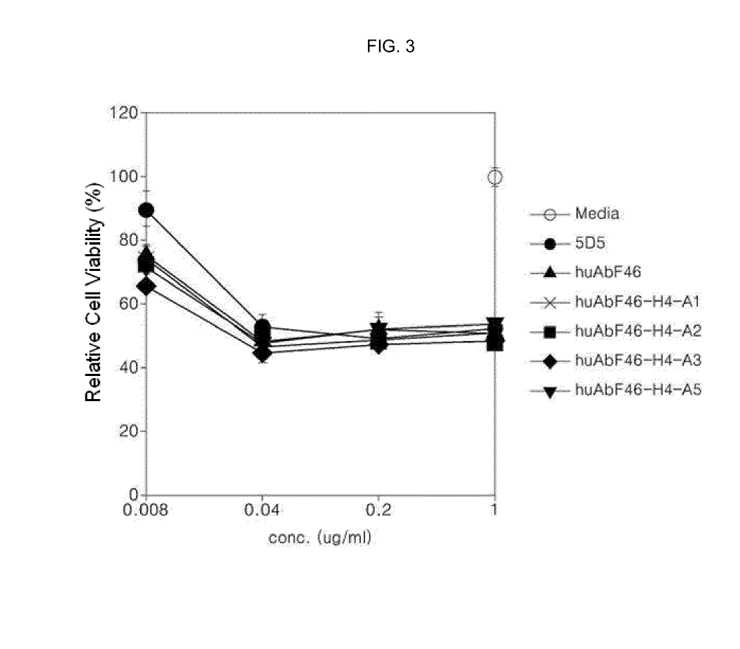

As shown in FIG. 3, relative cell viability of the antibody (huAbF46) in which the affinity was not improved was 77% at the lowest concentration of 0.008 ug/ml, and relative cell viabilities of antibodies having improved affinities, i.e., huAbF46-H4-A1, huAbF46-H4-A2, and huAbF46-H4-A5 were respectively 74, 73, and 72% similar to each other. The relative cell viability of huAbF46-H4-A3, at 66%, was considerably decreased. In addition, at 0.04 ug/ml, where the effect on cell viability is maximized, relative cell viabilities of all of the 4 types of antibodies were equal or less than that of the antibody 5D5 (53%). Accordingly, as a result of improving affinity, efficiency and safety were significantly improved compared to the control.

(3) Akt Phosphorylation

Cellular processes regulated by Akt include cell proliferation, cell survival, cell size control, and response to nutrient availability, intermediary metabolism, angiogenesis, and tissue invasion. All these processes represent characteristics of cancer and many oncoproteins and tumor suppressors intersect in the Akt pathway, finely regulating cellular functions at the interface of signal transduction and classical metabolic regulation. Thus, as the content of phosphorylated Akt that is an active form increases, the activity of cancer cells increases. Here, the degree of inhibiting Akt phosphorylation by the 4 types of antibodies having improved affinities was evaluated.

To compare agonism of the 4 types of antibodies having improved affinities, as prepared in Example 5, Caki-1 cells (Korean Cell Line Bank) were used to confirm the degree of Akt phosphorylation. Mouse IgG was used as a negative control, and antibody 5D5 (a known agonist) was used as a positive control.

2×10⁵ cells/ml of Caki-1 cells were introduced to a 96-well plate, and after 24 hours, each of 5 ug/ml of the antibodies was treated in serum free medium for 30 minutes. Lysis of the cells treated with the antibodies was performed and the degree of Akt phosphorylation was measured using a PathScan phospho-AKT1 (Ser473) chemiluminescent Sandwich ELISA kit (Cell Signaling, cat. no #7134S).

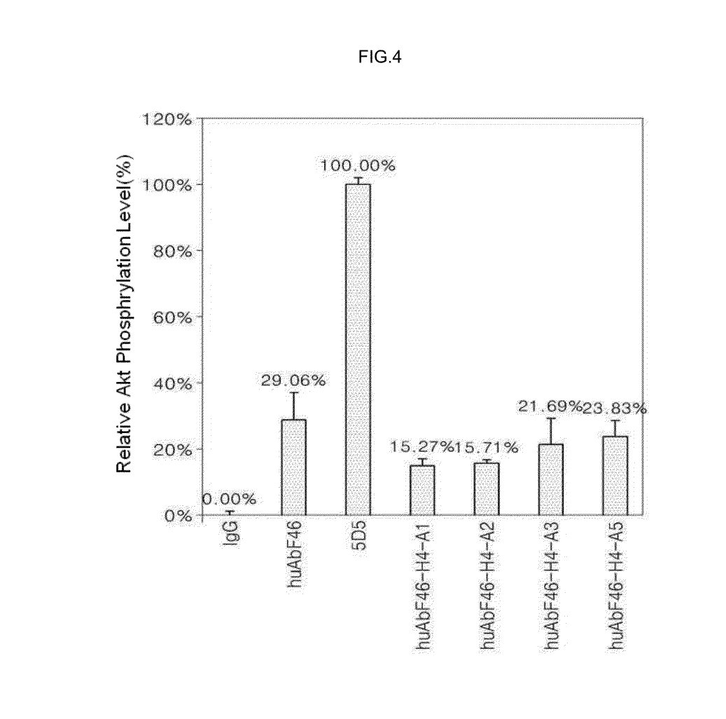

As shown in FIG. 4, it was identified that the degree of inhibiting Akt phosphorylation of all of the 4 types of antibodies was improved. In particular, the degrees of Akt phosphorylation of huAbF46-H4-A1 and huAbF46-H4-A2 were 15.27% and 15.71%, respectively, which were about 49% of that (29.06%) before affinity was improved (huAbF46). Thus, it was identified that safety of the affinity maturated antibodies were considerably improved. In contrast, antibody 5D5 exhibits very high relative Akt phosphorylation level (100%), indicating that antibody 5D5 shows very high agionism and very low safety.

(4) Identification of Degree of Degradation of c-Met

In order to identify anti-cancer effects of the 4 types of antibodies having improved affinities, as prepared in Example 5, the degree of degradation of c-Met bound to the antibodies was evaluated. A relative total amount of c-Met was obtained by measuring the change in the total amount of c-Met after the antibody binds to c-Met to degrade c-Met via internalization, and thus the efficacy of the antibody was evaluated.

MKN45 cells (2×10⁵ cells/ml) and each of the antibodies (5 ug/ml) were simultaneously introduced to a 96-well plate and incubated for 24 hours. Then, lysis of the cells treated with antibodies was performed and the change in the total amount of c-Met was measured using a Human total HGF R/c-Met ELISA KIT (R&D systems, DYC358) and analyzed.

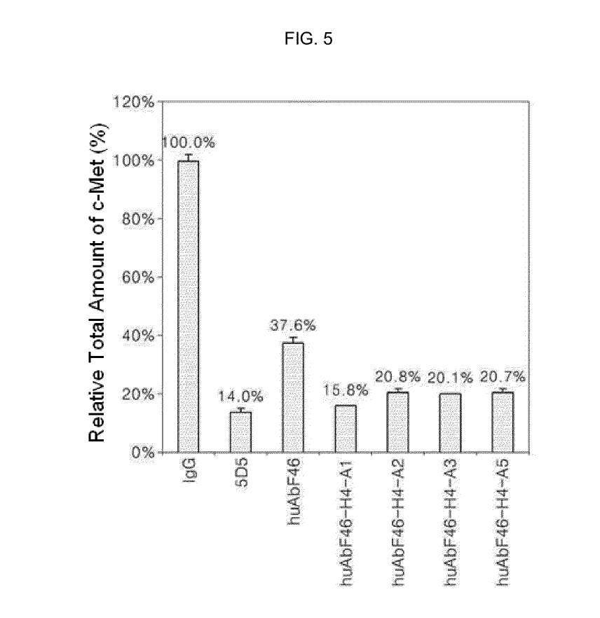

As a result, referring to FIG. 5, it was identified that the degree of degradation of c-Met was improved when cells were treated with the 4 types of antibodies having improved affinities compared to cells treated with the huAbF46 antibody. The degree of degradation of c-Met in cells treated with huAbF46-H4-A1 was increased by about 37% compared to cells treated with huAbF46. The degrees of degradation of c-Met in cells treated with huAbF46-H4-A2, huAbF46-H4-A3, and huAbF46-H4-A5 were increased by about 28% compared to cells treated with huAbF46. As shown in FIGS. 4 and 5, the affinity maturated antibodies show equal or higher degree of degradation of c-Met as well as very higher safety compared to those of antibody 5D5.

### Example 10

### Analysis of In Vivo Biological Activity of Selected Antibodies Having Improved Affinities

In order to identify anti-cancer effects of the 4 types of antibodies having improved affinities, as prepared in Example 5, a decrease in the size of tumor cells in a brain cancer or gastric cancer mouse xenograft model transplanted with U87MG brain cancer cells (Korean Cell Line Bank) or MKN45 gastric cancer cells (Japanese Cancer Research Bank, JCRB, Tokyo, Japan) was observed when the antibodies having improved affinities were administered thereto in vivo.

For MKN45 model (FIG. 6A), 5×10⁶ MKN45 cells (100 uL) were administered via subcutaneous injection to 6 week-old male BALB/C nude mice (SLAC Laboratories, Shanghai, China). For U87MG model (FIG. 6B), 2.5×10⁶ U87MG cells were administered. One wee after the tumor inoculation, the mice were randomized into Vehicle (PBS) or huAbF46-H4-A1 treatment groups at a various doses (0.2 mg/kg-10 mg/kg). Each group consisted of 15 mice. For MKN45 model, each treatment was given once a week via intravenous route, for total of 4 doses. For U87MG model, the treatment was given every 10 days for total of 3 doses.

Referring to FIGS. 6A and 6B, in both the U87MG brain cancer and the MKN45 gastric mouse cancer models, dose-dependent tumor growth inhibiting effects of huAbF46-H4-A1 were identified.

### Example 11

### Preparation of huAbF46-H4-A1 Having Replaced Constant Region and/or Hinge Region

Among the selected 4 types of antibodies, huAbF46-H4-A1 was determined to have the highest binding affinity to c-Met and the lowest degrees of Akt phosphorylation and c-Met differentiation. The hinge region, or the constant region and hinge region, of huAbF46-H4-A1 was replaced.

An antibody including a heavy chain that includes a heavy chain variable region of huAbF46-H4-A1, a U6-HC7 hinge, and a human IgG1 constant region, and a light chain that includes a light chain variable region of huAbF46-H4-A1 and a human kappa constant region was named huAbF46-H4-A1(U6-HC7), an antibody including a heavy chain that includes a heavy chain variable region of huAbF46-H4-A1, a human IgG2 hinge, and a human IgG1 constant region and a light chain that includes a light chain variable region of huAbF46-H4-A1 and a human kappa constant region was named huAbF46-H4-A1 (IgG2 hinge), and an antibody including a heavy chain that includes a heavy chain variable region of huAbF46-H4-A1, a human IgG2 hinge, and a human IgG2 constant region and a light chain that includes a light chain variable region of huAbF46-H4-A1 and a human kappa constant region was named huAbF46-H4-A1 (IgG2 Fc). In addition, in order to increase productivity of the 3 types of antibodies all histidine was replaced with tyrosine at position 36 in the light chain including the human kappa constant region.

In order to prepare the 3 types of antibodies, a polynucleotide (SEQ ID NO: 63) encoding a polypeptide (SEQ ID NO: 62) including the heavy chain variable region of huAbF46-H4-A1, the U6-HC7 hinge region, and the human IgG1 constant region, a polynucleotide (SEQ ID NO: 65) encoding a polynucleotide (SEQ ID NO: 65) including the heavy chain variable region of huAbF46-H4-A1, the human IgG2 hinge region, and the human IgG1 constant region, a polynucleotide (SEQ ID NO: 67) encoding a polypeptide (SEQ ID NO: 66) including the heavy chain variable region of huAbF46-H4-A1, the human IgG2 hinge region, and the human IgG2 constant region, and a polynucleotide (SEQ ID NO: 69) encoding a polypeptide (SEQ ID NO: 68) including the light chain variable region of huAbF46-H4-A1 in which histidine is replaced with tyrosine at position 36 and the human kappa constant region were synthesized by Bioneer, Inc. Then, vectors for expression of the antibodies were constructed by cloning a DNA fragment having the nucleic acid sequence corresponding to the heavy chain in a pOptiVEC™-TOPO TA Cloning Kit included in an OptiCHO™ Antibody Express Kit (Cat No. 12762-019) manufactured by Invitrogen, and a DNA fragment having the nucleic acid sequence corresponding to the light chain in a pcDNA™3.3-TOPO TA Cloning Kit (Cat No. 8300-01).

The constructed vectors were amplified using a Qiagen Maxiprep kit (Cat No. 12662), and vectors including the heavy chain and vectors including the light chain were added to 293T cells (2.5×10⁷) at a ratio of about 4:1 (about 80 ug:20 ug) with 360 ul of 2 M CaCl₂ and were transfected. Then, the mixture was cultured in a DMEM medium added with 10% (w/v) FBS at 37° C. in 5% (v/v) CO₂ conditions for 5 hours, and then cultured in a DMEM medium without FBS at 37° C. in 5% (v/v) CO₂ conditions for 48 hours.

The cultured cells were centrifuged, and 100 ml of each supernatant was purified using AKTA Prime (GE healthcare). Protein A column (GE healthcare, 17-0405-03) was placed in the AKTA Prime, and the cultured solution was flowed at a flow rate of 5 ml/min and was eluted with IgG elution buffer (Thermo Scientific, 21004). The buffer was exchanged with a PBS buffer, and 3 types of antibodies (huAbF46-H4-A1 (U6-HC7), huAbF46-H4-A1(IgG2 hinge), and huAbF46-H4-A1(IgG2 Fc)) were purified.

In addition, another light chain (SEQ ID NO: 73) was prepared by replacing the amino acid residue, serine, at the position 27e (according to kabat numbering) of the light chain variable region of huAbF46-H4-A1 with tryptophan. Then, an antibody including the prepared light chain and the heavy chain of huAbF46-H4-A1 (IgG2 Fc) was prepared as described above, and named as L3-11Y. The binding affinity of the L3-11Y antibody was measured according to the method described in Example 8, and the measured binding affinity (K_{D} (nM)) was less than 0.01 (<0.01).

### Example 12

### Analysis of In Vitro Biological Activity of huAbF46-H4-A1 Having Replaced Constant Region and/or Hinge Region

(1) In Vitro Cell Proliferation Analysis

In order to identify anti-cancer effects of the three types of antibodies prepared in Example 11, in vitro cell proliferation analysis was performed using MKN45 gastric cancer cells having c-Met on the cell membrane (Japanese Cancer Research Bank, JCRB, Tokyo, Japan).

1×10⁴ MKN45 cells suspended in 50 ul of 5% (w/v) FBS/DMEM culture medium were introduced to each well of a 96-well plate. Then, the cells were either not treated or were treated with 50 ul of the 3 types of antibodies at a concentration of 0.008, 0.04, 0.2, or 1 ug/ml. After incubating for 72 hours, the number of cells was quantified by using a CellTiter-Glo Luminescent Cell Viability Assay Kit (Promega, G7570) with a leuminometer (PerkinElmer, 2104 Multilabel reader).

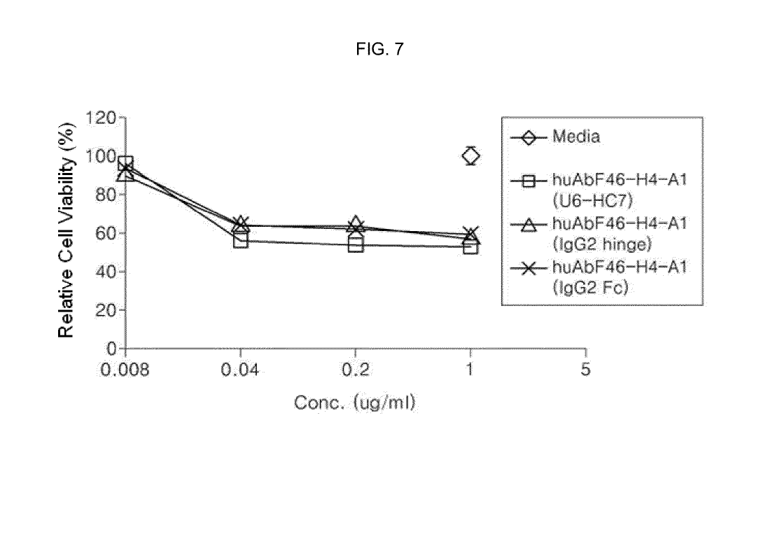

As shown in FIG. 7, when the 3 types of antibodies, huAbF46-H4-A1 (U6-HC7), huAbF46-H4-A1 (IgG2 hinge), and huAbF46-H4-A1 (IgG2 Fc), were treated at a concentration of 0.04 ug/ml or less, the relative cell viability was about 60%.

(2) Akt Phosphorylation

To compare agonism of the 3 types of antibodies having improved affinities, as prepared in Example 11, Caki-1 cells (Korean Cell Line Bank) were used to confirm the degree of Akt phosphorylation. Mouse IgG was used as a negative control, and a 5D5 antibody (a known agonist) was used as a positive control.

2×10⁵ cells/ml of Caki-1 cells were introduced to a 96-well plate, and after 24 hours, each of 5 ug/ml of the antibodies was treated in serum free medium for 30 minutes. Lysis of the cells treated with antibodies was performed and a degree of Akt phosphorylation inhibition was measured using a PathScan phospho-AKT1 (Ser473) a chemiluminescent Sandwich ELISA kit (Cell Signaling, cat. no #7134S).

The obtained results are shown in FIG. 8A. As shown in FIG. 8A, the degrees of inhibiting Akt phosphorylation of all of the 3 types of antibodies were 18% or less. Thus, it was identified that safety was considerably improved.

In addition, the degrees of Akt phosphorylation inhibition of huAbF46-H4-A1(IgG2 Fc) and L3-11Y were also measured according the above method. The obtained results are shown in FIG. 8B. As shown in FIG. 8B, L3-11Y exhibits an equal or higher activity of Akt phosphorylation inhibition compared to that of huAbF46-H4-A1(IgG2 Fc).

(3) Identification of Degree of Degradation of c-Met

In order to identify anti-cancer effects of the 3 types of antibodies having improved affinities, as prepared in Example 11, the degree of degradation of c-Met bound to the antibody was evaluated. MKN45 cells (2×10⁵ cells/ml) and each of the antibodies (5 ug/ml) were simultaneously introduced to a 96-well plate and incubated for 24 hours. Then, lysis of the cells treated with antibodies was performed and a change of the total amount of c-Met was measured using a Human total HGF R/c-Met ELISA KIT (R&D systems, DYC358) and analyzed.

The obtained results are shown in FIG. 9A. As shown in FIG. 9A, it was identified that the degree of degradation of c-Met was improved in cells treated with the 3 types of antibodies having improved affinities compared to cells treated with the huAbF46 antibody.

In addition, the degrees of c-Met degradation of huAbF46-H4-A1 (IgG2 Fc) and L3-11Y were also measured according the above method. The obtained results are shown in FIG. 9B. As shown in FIG. 9B, L3-11Y exhibits an approximately equal activity of c-Met degradation compared to that of huAbF46-H4-A1 (IgG2 Fc).

### Example 13

### Analysis of In Vivo Biological Activity of huAbF46-H4-A1 Having Replaced Constant Region and/or Hinge Region

In order to identify anti-cancer effects of the 3 types of antibodies having improved affinities, as prepared in Example 11, a decrease in the size of tumor cells in a brain cancer or gastric cancer mouse xenograft model transplanted with U87MG brain cancer cells (Korean Cell Line Bank) or MKN45 gastric cancer cells (Japanese Cancer Research Bank, JCRB, Tokyo, Japan) was observed when the antibodies having improved affinities were administered in vivo.

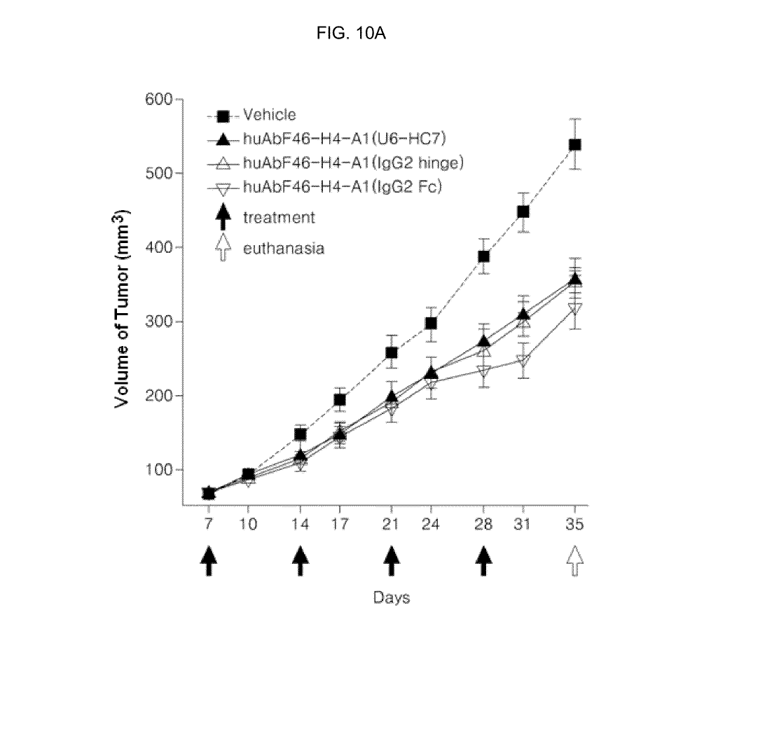

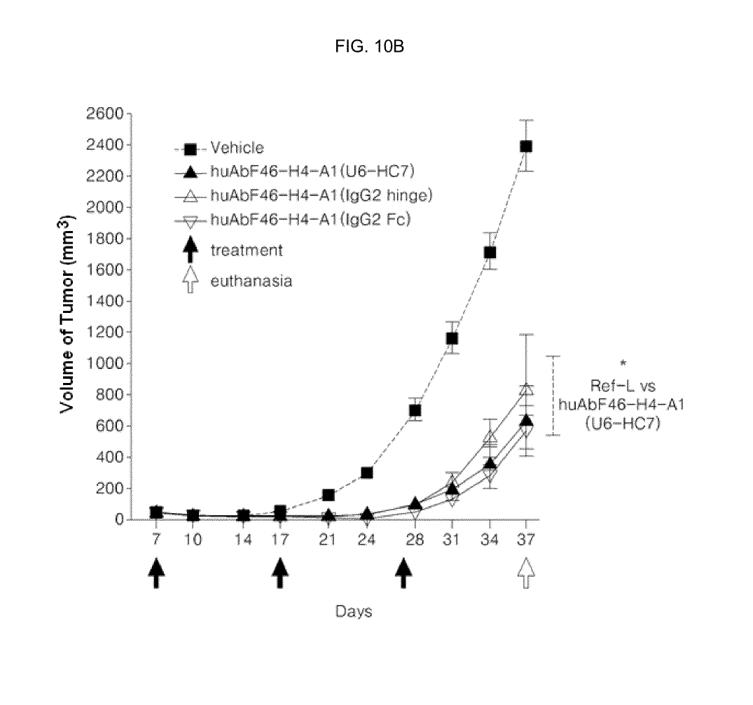

For MKN45 model (FIG. 10A), 5×10⁶ MKN45 cells (100 uL) were administered via subcutaneous injection to 6 week-old male BALB/C nude mice (SLAC Laboratories, Shanghai, China). For U87MG model (FIG. 10B), 2.5×10⁶ U87MG cells were administered. One week after the tumor inoculation, the mice were randomized into Vehicle (PBS) group or 3 different antibody treatment groups (huAbF46-H4-A1 U6-HC7, IgG2 hinge, or IgG2 Fc). Each group consisted of 15 mice. For MKN45 model, each treatment was given at 1 mg/kg once a week via intravenous route, for total of 4 doses. For U87MG model, the treatment was given at 0.2 mg/kg every 10 days for total of 3 doses.

In both of the MKN45 gastric cancer (FIG. 10A) or U87MG brain cancer mouse cancer models (FIG. 10B), the three types of antibodies showed comparable levels of tumor growth inhibiting effect.

### Example 14

### Anti-Migration Activity of huAbF46-H4-A1(IgG2 Fc) (In Vitro)

Cell migration inhibition ratio of huAbF46-H4-A1(IgG2 Fc) was analyzed by RTCA (Real Time Cell Analyzer). RTCA is a labeling-free cell-based assay system integrating microelectronics and cell biology, suitable for uninterrupted monitoring of biological processes of living cells.

NCI-H441 cells (ATCC Cat. # HTB-174), SNU-638 cells (Korean Cell Line Bank (KCLB), Cat. #00638), and Capan-2 cells (ATCC Cat. # HTB-80) were respectively plated at a density of 1×10⁵ cells per well in 130 μl of serum-free RPMI 1640 medium onto upper chamber of a 16-well CIM plate (Roche). To test whether huAbF46-H4-A1(IgG2 Fc) induces cell migration inhibition, 10 μg/mL of huAbF46-H4-A1 (IgG2 Fc) were treated in lower chamber in the absence or presence of HGF (200 ng/mL) in FBS 10% (v/v) RPMI 1640 medium (total volume of 160 μL).

During incubation at 37° C. with 5% CO₂, the cell Index (CI) was recorded in real time. The obtained results were summarized in Table 4.

TABLE 4

| | Cell Line | | | | | |
|---|---|---|---|---|---|---|
| | H441 | | SNU-638 | | Capan-2 | |
| Conc. (μg/mL) | 10 | | 10 | | 10 | |
| Time point (hr) | 20 | | 48 | | 35 | |
| HGF (200 ng/mL) | (+) | (−) | (+) | (−) | (+) | (−) |
| Relative inhibition rate | 69.8 | N/A | 73.4 | 10.7 | 86.2 | Not tested |
| (%) | | | | | | |

cf. N/A: cells did not migrate in the absence of hepatocyte growth factor (HGF)

huAbF46-H4-A1(IgG2 Fc) showed dose-dependent anti-migration activity, using Real Time Cell Analyzer (RTCA), in 3 cancer cell lines. The relative migration inhibition rate shown in Table 4 was calculated at a specific time-point when the inhibition level was most significant per cell line. The ‘N/A’ mark in Table 4 means that cells did not migrate in the absence of hepatocyte growth factor (HGF). Therefore, the inhibitory level of huAbF46-H4-A1(IgG2 Fc) could not be measured under these conditions.

### Example 15

### Anti-Migration Activity of huAbF46-H4-A1(IgG2 Fc) (In Vivo)

For the MKN45 orthotopic xenograft experiment, human gastric cancer MKN45 cells (Japanese Cancer Research Bank, JCRB, Tokyo, Japan) were inoculated into donor BALB/C nude mice. When the tumor size reached the size of 600-800 mm3, the donor mice were euthanized, and the tumor was excised through sterile surgical procedure. The tumors were cut into fragments the size of 1×1×1 mm and implanted into the wall of great gastric curvature of recipient mice through sterile surgery under isoflurane anesthesia. 7 days after the surgery, recipient mice with orthotopic tumors were randomized into groups according to body weight and dosing was commenced. huAbF46-H4-A1(IgG2 Fc) was injected into a vein once a week for 9 weeks. At the end of the in vivo study, orthotopic tumor weight was recorded and metastasis to the other organ was checked by macroscopic examination.

The number of metastasis and adhesion lesion in MKN45 orthotopic xenograft are shown in Table 5:

TABLE 5

| | Number of metastasis |
|---|---|
| Groups | lesion |
| PBS | 4 |
| 5-FU | 0 |
| huAbF46-H4-A1(IgG2 Fc) 10 mg/kg | 0 |
| huAbF46-H4-A1(IgG2 Fc) 5 mg/kg | 0 |
| huAbF46-H4-A1(IgG2 Fc) 1 mg/kg | 1 |

As shown in Table 5, metastasis from stomach to the other organ, such as liver and kidney was found 4 of 9 mice in the vehicle group, whereas occurrence of metastasis decreased with huAbF46-H4-A1(IgG2 Fc) treatment in a dose-dependent manner. In conclusion, huAbF46-H4-A1(IgG2 Fc) showed an anti-tumor efficacy in MKN45 orthotopic xenograft in dose dependent manner and inhibited the occurrence of metastasis to the other organ.

### Example 16

### Anticancer Effect of huAbF46-H4-A1(IgG2 Fc) on EBC1 Cell Line

16.1. Experiment 1

The effect of huAbF46-H4-A1(IgG2 Fc) on the growth of human lung cancer cell line EBC-1 subcutaneous xenografts in BALB/C nude mice was evaluated. 5 million EBC-1 cells were injected s.c. Four days after inoculation, dosing commenced in treatment groups. For the xenograft experiment, EBC-1 cells (JCRB, Japan) were subcutaneously inoculated into donor BALB/C nude mice. Each group consisted of 15 animals. Vehicle control (PBS) and huAbF46-H4-A1(IgG2 Fc) (0.03, 0.1, 0.3, 1, 3 or 10 mg/kg) treatment groups were dosed i.v. q.w.×4 injections. Tumor volumes and body weights were measured two to three times a week for total study period about 4 weeks. The tumor volume (V) was calculated as follows: V (mm3)=[long axis length (mm)×(short axis length (mm))²]/2.

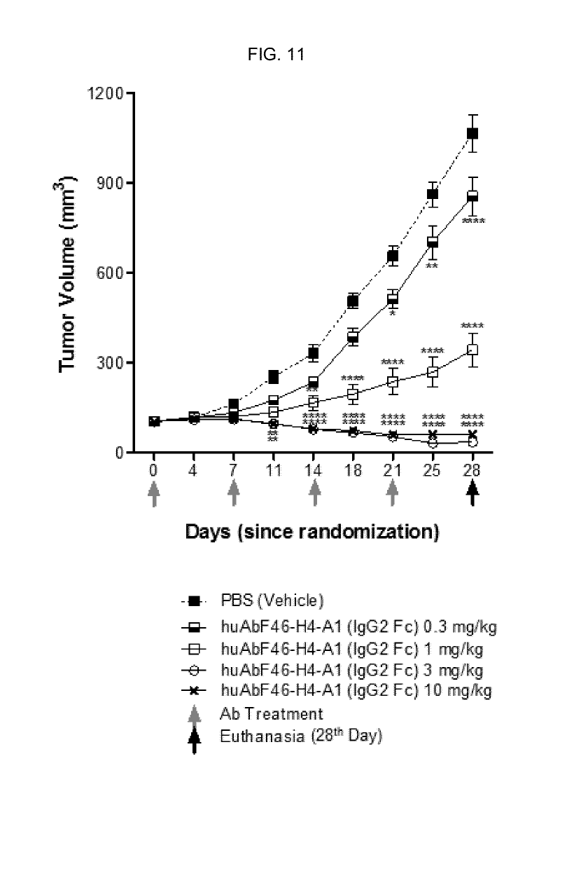

The obtained results (tumor volumes) are shown in FIG. 11. In FIG. 11, tumor volumes were measured on indicated days are plotted (mean and s.e.m.) for treatment groups (huAbF46-H4-A1(IgG2 Fc)) and PBS (negative control) group. Asterisks (*) represent P-values versus vehicle group according to repeated measures ANOVA on each indicated day (*P<0.05, **P<0.01, ***P<0.001, ****P<0.0001). As shown in FIG. 11, the tumor volumes are significantly reduced by treatment of huAbF46-H4-A1(IgG2 Fc) in dose-dependent manner.

16.2. Experiment 2

To study the effect of anti-c-Met antibodies on tumor growth in vivo, tumor xenograft studies were performed using 5-6 weeks old male BALB/c Nude mice. Mice were acclimated for at least a week before they received tumor inoculation. 5 million EBC1 cells (JCRB, Japan) in 200 μl of serum-free media/matrigel (50:50 v/v) were injected subcutaneously into the right flank region of the mice under anesthesia by 1-2% isoflurane. After 7 days, when the average tumor size was close to 100-200 mm³, mice were randomized into the following treatment groups: 5D5 (5 mg/kg I.V. once a week), huAbF46-H4-A1(IgG2 Fc) (5 mg/kg I.V. once a week), and vehicle (PBS 0.2 ml I.V. once a week). Each treatment group consisted of 15 mice. Tumor volumes and body weights were measured two to three times a week for total study period about 4 weeks. The tumor volume (V) was calculated as follows: V (mm³)={long axis length (mm)×(short axis length (mm))²}/2. Tumor growth inhibition was calculated as follows: 100−100*(ΔTV in huAbF46-H4-A1(IgG2 Fc) group)/(ΔTV in Vehicle group), where ΔTV=TV(end)−TV(d0).

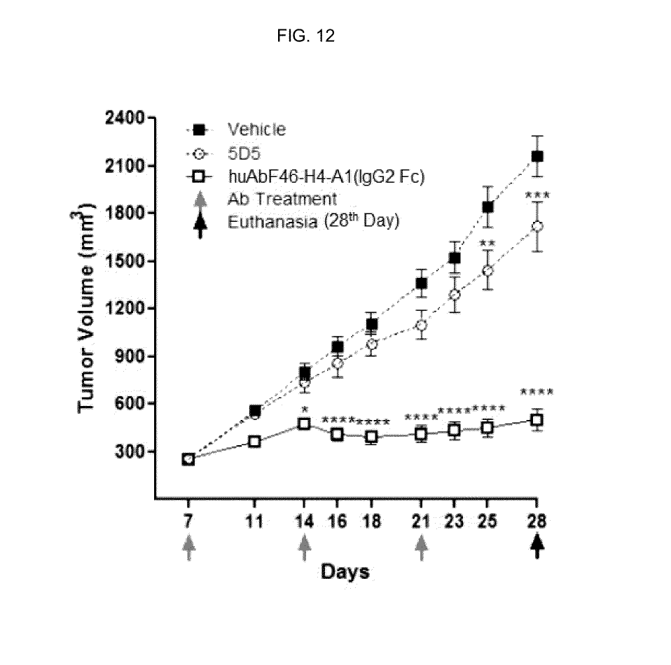

The obtained results are shown in FIG. 12. In FIG. 12, tumor volumes measured on indicated days are plotted (mean and SEM) for two treatment groups (5D5 and huAbF46-H4-A1(IgG2 Fc)) and vehicle (negative control) group. Asterisks (*) represent p values versus vehicle group according to repeated measures ANOVA (*: p<0.05, **: p<0.01, ***: p<0.001, ****: p<0.0001). As shown in FIG. 12, huAbF46-H4-A1 (IgG2 Fc) demonstrated a strong inhibition of tumor growth, resulting in the tumor growth inhibition of 77% in EBC1 model. In comparison, 5D5 treatment resulted in much less tumor growth inhibition.

### Example 17

### Anticancer Effect of huAbF46-H4-A1(IgG2 Fc) on MHCC97H Cell Line

The antitumor activity of huAbF46-H4-A1(IgG2 Fc) was evaluated in a BALB/C nude mouse MHCC97H (human liver cancer cell line) xenograft model (referring to Example 16.2). Approximately 3 million MHCC97H cells in 100 μL of serum-free media were injected via s.c. to each of the 140 mice under anesthesia by 1-2% isoflurane. Ten days after subcutaneous inoculation with MHCC97H tumor cells, dosing commenced in the following treatment groups: 0 (PBS vehicle), 0.2, 1, 5 or 10 mg/kg huAbF46-H4-A1 (IgG2 Fc) i.v., q.w.×4 weeks; 30 mg/kg sorafenib p.o., q.d.×4 weeks (positive control). Each group consisted of 15 mice. Tumor volumes and body weights were measured two to three times a week for total study period about 4 weeks. The tumor volume (V) was calculated as follows: V (mm³)={long axis length (mm)×(short axis length (mm))²}/2.

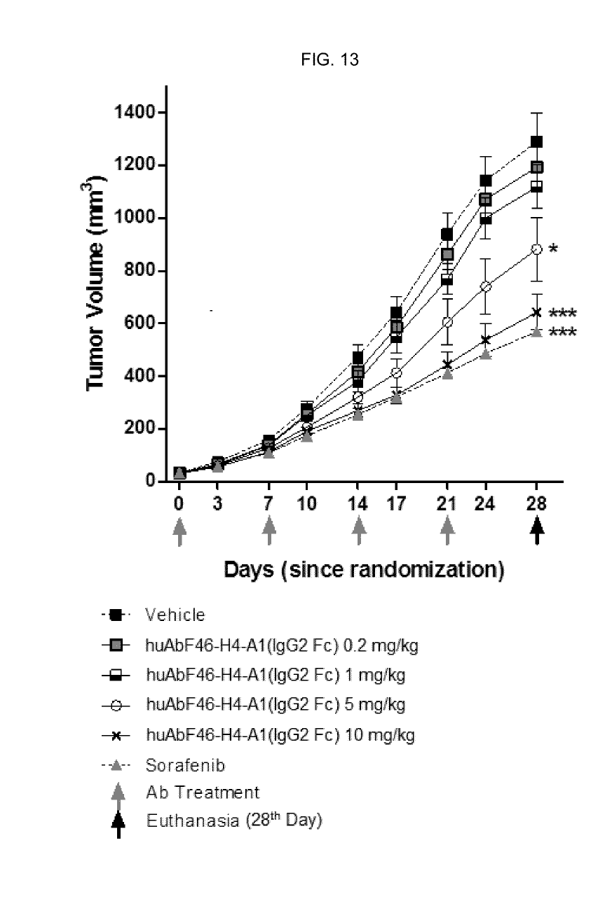

The obtained results are shown in FIG. 13. In FIG. 13, tumor volumes were measured on indicated days are plotted (mean and s.e.m.) for treatment groups (huAbF46-H4-A1(IgG2 Fc)) and vehicle (negative control) group. Asterisks (*) represent P-values versus vehicle group according to repeated measures ANOVA, plotted for the last day only (*P<0.05, **P<0.01, ***P<0.001, ****P<0.0001). As shown in FIG. 13, treatment with huAbF46-H4-A1(IgG2 Fc) 5 mg/kg significantly inhibited tumor growth starting from the 14^{th} day (p<0.05) and throughout the remainder of the study (p<0.01). Treatment with huAbF46-H4-A1(IgG2 Fc) 10 mg/kg significantly inhibited tumor growth starting from the 3^{rd} day (p<0.05) and throughout the remainder of the study (p<0.01). Treatment with huAbF46-H4-A1(IgG2 Fc) at 5 and 10 mg/kg also significantly reduced tumor weight (p<0.01 or p<0.05, respectively) compared to the PBS vehicle group. The positive control drug sorafenib at 30 mg/kg given daily and huAbF46-H4-A1(IgG2 Fc) at 5 and 10 mg/kg demonstrated significant inhibition of MHCC97H tumor growth, whereas other treatments resulted in no significant inhibitory effect on tumor growth. Furthermore, huAbF46-H4-A1(IgG2 Fc) treatment showed a dose-response relationship with respect to tumor volume, relative tumor volume and tumor weight reductions compared to the control group. In conclusion, treatment with huAbF46-H4-A1(IgG2 Fc) showed anti-tumor efficacy in this human liver cancer MHCC97H xenograft model.

### Example 18

### Anticancer Effect of huAbF46-H4-A1(IgG2 Fc) on PDT Cell Line

Tumor xenograft study using patient-derived tumor (PDT; NSCLC and RCC) was performed using 5-7 weeks old male NRMI nu/nu mice. The tumor fragments passaged in vivo in donor mice were collected, made into equally-sized fragments, and implanted subcutaneously into the flank region of the recipient mice under anesthesia. When the average tumor size was 50-250 mm3, mice were randomized into either huAbF46-H4-A1(IgG2 Fc) (5 mg/kg I.V. once a week) treatment group, or vehicle (PBS I.V. once a week) treatment group. Each group consisted of 10 mice. Tumor volumes and body weights were measured two to three times a week for total study period about 6 weeks. The tumor volume (V) was calculated as follows: V (mm3)=[long axis length (mm)×(short axis length (mm))²]/2. At the end of the in vivo phase, the mice were euthanized; tumors were extracted and fixed in 10% formaldehyde or frozen for further analysis. Tumor growth inhibition was calculated as follows: 100−100*(ΔTV in huAbF46-H4-A1(IgG2 Fc) group)/(ΔTV in Vehicle group), where ΔTV=TV(end)−TV(d0).

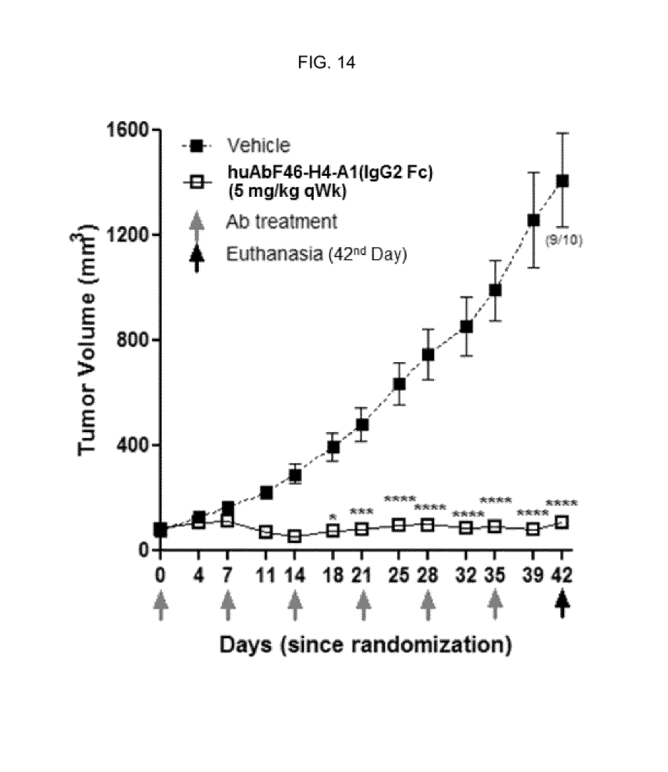

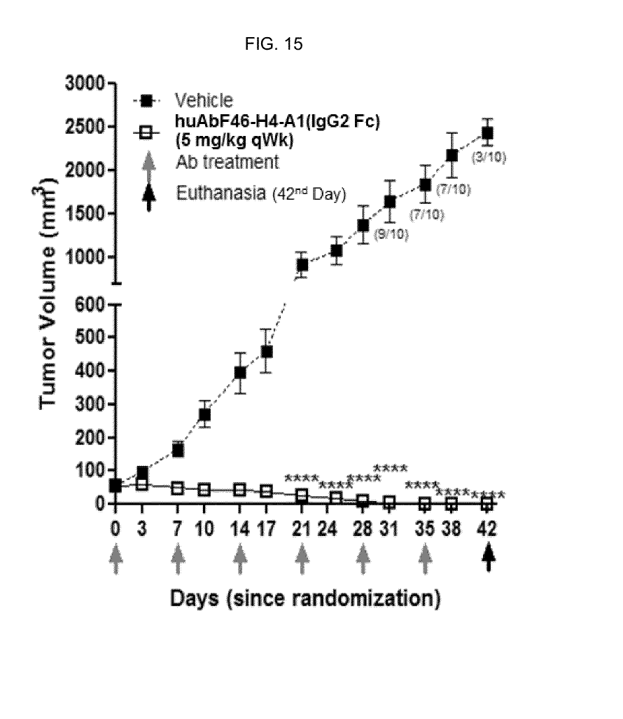

The obtained results are shown in FIGS. 14 (NSCLC) and 15 (RCC). In FIGS. 14 and 15, tumor volumes were measured on indicated days are plotted (mean and SEM) for the two groups (Vehicle or huAbF46-H4-A1(IgG2 Fc)). Asterisks (*) represent p values versus Vehicle group according to repeated measures ANOVA (*: p<0.05, **: p<0.01, ***: p<0.001, ****: p<0.0001). Numbers inside parenthesis shows remaining mice at each time point, as mice were euthanized before the end of the study when TV reached 2000 mm³. As shown in FIGS. 14 and 15, huAbF46-H4-A1(IgG2 Fc) showed very potent and statistically significant tumor growth inhibition in the PDT model. huAbF46-H4-A1(IgG2 Fc) caused tumor growth arrest, and even complete regression, in these models. In these examples (one NSCLC model (FIG. 14), one RCC model (FIG. 15)), huAbF46-H4-A1(IgG2 Fc) treatment resulted in tumor volume inhibitions of 100% and 98%, respectively.

As described above, according to the anti-c-Met antibody and the pharmaceutical composition for preventing or treating cancer including the same according to one or more embodiments of the present invention, cancer may be effectively prevented or treated.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of prevention or treatment of a cancer comprising administering an anti-c-Met or an antigen-binding fragment thereof to a subject in need of prevention or treatment of the cancer.

2. The method of claim 1, wherein the anti-c-Met antibody or antigen-binding fragment thereof comprising:
a heavy chain variable region comprising at least one heavy chain complementarity determining region (CDR) selected from the group consisting of CDR-H1 having an amino acid sequence of SEQ ID NO: 1 or SEQ ID NO: 4, CDR-H2 having an amino acid sequence of SEQ ID NO: 2 or SEQ ID NO: 5, and CDR-H3 having an amino acid sequence of SEQ ID NO: 3 or SEQ ID NO: 6; and
a light chain variable region comprising at least one light chain CDR selected from the group consisting of CDR-L1 having an amino acid sequence of SEQ ID NO: 7, CDR-L2 having an amino acid sequence of SEQ ID NO: 8, and CDR-L3 having an amino acid sequence of SEQ ID NO: 9, wherein SEQ ID NOS: 4 to 9 are respectively represented by following Formula I to VI:
Xaa₁-Xaa₂-Tyr-Tyr-Met-Ser (SEQ ID NO: 4), wherein Xaa₁ is Pro or Ser or is absent, and Xaa₂ is Glu or Asp; Formula I
Arg-Asn-Xaa₃-Xaa₄-Asn-Gly-Xaa₅-Thr (SEQ ID NO: 5), wherein Xaa₃ is Asn or Lys, Xaa₄ is Ala or Val, and Xaa₅ is Asn or Thr; Formula II
Asp-Asn-Trp-Leu-Xaa₆-Tyr (SEQ ID NO: 6); wherein Xaa₆ is Ser or Thr; Formula III
Lys-Ser-Ser-Xaa₇-Ser-Leu-Leu-Ala-Xaa₈-Gly-Asn-Xaa₉-Xaa₁₀-Asn-Tyr-Leu-Ala (SEQ ID NO: 7), wherein Xaa₇ is His, Arg, Gln, or Lys, Xaa₈ is Ser or Trp, Xaa₉ is His or Gln, and Xaa₁₀ is Lys or Asn; Formula IV
Trp-Xaa₁₁-Ser-Xaa₁₂-Arg-Val-Xaa₁₃ (SEQ ID NO: 8), wherein Xaa₁₁ is Ala or Gly, Xaa₁₂ is Thr or Lys, and Xaa₁₃ is Ser or Pro; and Formula V
Xaa₁₄-Gln-Ser-Tyr-Ser-Xaa₁₅-Pro-Xaa₁₆-Thr (SEQ ID NO: 9), wherein Xaa₁₄ is Gly, Ala, or Gln, Xaa₁₅ is Arg, His, Ser, Ala, Gly, or Lys, and Xaa₁₆ is Leu, Tyr, Phe, or Met. Formula VI

3. The method of claim 2, wherein,
the CDR-H1 a polypeptide having an amino acid sequence of SEQ ID NO: 22, 23, or 24; the CDR-H2 is a polypeptide having an amino acid sequence of SEQ ID NO: 25 or 26.; the CDR-H3 is a polypeptide having an amino acid sequence of SEQ ID NO: 27 or 28; the CDR-L1 is a polypeptide having an amino acid sequence of SEQ ID NO: 29, 30, 31, 32, 33, or 71; the CDR-L2 is a polypeptide having an amino acid sequence of SEQ ID NO: 34, 35, or 36; or the CDR-L3 is a polypeptide having an amino acid sequence of SEQ ID NO: 13, 14, 15, 16, or 37.

4. The method of claim 1, wherein the anti-c-Met antibody or antigen-binding fragment thereof comprising:
a heavy chain variable region comprising at least one heavy chain complementarity determining region (CDR) selected from the group consisting of CDR-H1 having an amino acid sequence of SEQ ID NO: 1, CDR-H2 having an amino acid sequence of SEQ ID NO: 2, and CDR-H3 having an amino acid sequence of SEQ ID NO: 3; and
a light chain variable region comprising at least one light chain CDR selected from the group consisting of CDR-L1 having an amino acid sequence of SEQ ID NO: 7, CDR-L2 having an amino acid sequence of SEQ ID NO: 8, and CDR-L3 having an amino acid sequence of SEQ ID NO: 9, wherein SEQ ID NOS: 7 to 9 are respectively represented by Formula IV to VI below:
Lys-Ser-Ser-Xaa₇-Ser-Leu-Leu-Ala-Xaa₈-Gly-Asn-Xaa₉-Xaa₁₀-Asn-Tyr-Leu-Ala (SEQ ID NO: 7), wherein Xaa₇ is His, Arg, Gln, or Lys, Xaa₈ is Ser or Trp, Xaa₉ is His or Gln, and Xaa₁₀ is Lys or Asn; Formula IV
Trp-Xaa₁₁-Ser-Xaa₁₂-Arg-Val-Xaa₁₃ (SEQ ID NO: 8), wherein Xaa₁₁ is Ala or Gly, Xaa₁₂ is Thr or Lys, and Xaa₁₃ is Ser or Pro; and Formula V
Xaa₁₄-Gln-Ser-Tyr-Ser-Xaa₁₅-Pro-Xaa₁₆-Thr (SEQ ID NO: 9), wherein Xaa₁₄ is Gly, Ala, or Gln, Xaa₁₅ is Arg, His, Ser, Ala, Gly, or Lys, and Xaa₁₆ is Leu, Tyr, Phe, or Met. Formula VI

5. The method of claim 4, wherein the anti-c-Met antibody or antigen-binding fragment thereof comprises the light chain variable region comprises at least one light chain CDR selected from the group consisting of CDR-L1 having an amino acid sequence of SEQ ID NO: 10 or 71, CDR-L2 having an amino acid sequence of SEQ ID NO: 11, and CDR-L3 having an amino acid sequence of SEQ ID NO: 13, 14, 15, or 16.

6. The method of claim 4, wherein the anti-c-Met antibody or antigen-binding fragment thereof comprises the heavy chain variable region has an amino acid sequence of SEQ ID NO: 17, and the light chain variable region has an amino acid sequence of SEQ ID NO: 18, 19, 20, 21, or 72.

7. The method of claim 1, wherein the anti-c-Met antibody or antigen-binding fragment thereof comprises:
a heavy chain comprising an amino acid sequence from 18^{th} to 462^{nd} of SEQ ID NO: 62, an amino acid sequence from 18^{th} to 461^{st} of SEQ ID NO: 64, or amino acid sequence from 18^{th} to 460^{th} of SEQ ID NO: 66, and a light chain comprising an amino acid sequence from 21^{st} to 220^{th} of SEQ ID NO: 68;
a heavy chain comprising an amino acid sequence from 18^{th} to 462^{nd} of SEQ ID NO: 62, an amino acid sequence from 18^{th} to 461^{st} of SEQ ID NO: 64, or amino acid sequence from 18^{th} to 460^{th} of SEQ ID NO: 66, and a light chain comprising an amino acid sequence from 21^{st} to 220^{th} of SEQ ID NO: 70; or
a heavy chain comprising an amino acid sequence from 18^{th} to 462^{nd} of SEQ ID NO: 62, an amino acid sequence from 18^{th} to 461^{st} of SEQ ID NO: 64, or amino acid sequence from 18^{th} to 460^{th} of SEQ ID NO: 66, and a light chain comprising an amino acid sequence of SEQ ID NO: 73.

8. The method of claim 1, wherein the anti-c-Met antibody or antigen-binding fragment thereof comprises an amino acid sequence of SEQ ID NO: 71, 72, or 73.

9. The method of claim 1, wherein the antibody or antigen-binding fragment is a monoclonal antibody, a mouse-derived antibody, a mouse-human chimeric antibody, or a humanized antibody,

10. The method of claim 1, wherein the antigen-binding fragment is scFv, (scFv)₂, Fab, Fab′, or F(ab′)₂.

11. A method of prevention or inhibition of metastasis of a cancer comprising administering an anti-c-Met or an antigen-binding fragment thereof to a subject in need of prevention or treatment of the cancer.

12. The method of claim 11, wherein the anti-c-Met antibody or antigen-binding fragment thereof comprising:
a heavy chain variable region comprising at least one heavy chain complementarity determining region (CDR) selected from the group consisting of CDR-H1 having an amino acid sequence of SEQ ID NO: 1 or SEQ ID NO: 4, CDR-H2 having an amino acid sequence of SEQ ID NO: 2 or SEQ ID NO: 5, and CDR-H3 having an amino acid sequence of SEQ ID NO: 3 or SEQ ID NO: 6; and
a light chain variable region comprising at least one light chain CDR selected from the group consisting of CDR-L1 having an amino acid sequence of SEQ ID NO: 7, CDR-L2 having an amino acid sequence of SEQ ID NO: 8, and CDR-L3 having an amino acid sequence of SEQ ID NO: 9, wherein SEQ ID NOS: 4 to 9 are respectively represented by following Formula I to VI:
Xaa₁-Xaa₂-Tyr-Tyr-Met-Ser (SEQ ID NO: 4), wherein Xaa₁ is Pro or Ser or is absent, and Xaa₂ is Glu or Asp; Formula I
Arg-Asn-Xaa₃-Xaa₄-Asn-Gly-Xaa₅-Thr (SEQ ID NO: 5), wherein Xaa₃ is Asn or Lys, Xaa₄ is Ala or Val, and Xaa₅ is Asn or Thr; Formula II
Asp-Asn-Trp-Leu-Xaa₆-Tyr (SEQ ID NO: 6); wherein Xaa₆ is Ser or Thr; Formula III
Lys-Ser-Ser-Xaa₇-Ser-Leu-Leu-Ala-Xaa₈-Gly-Asn-Xaa₉-Xaa₁₀-Asn-Tyr-Leu-Ala (SEQ ID NO: 7), wherein Xaa₇ is His, Arg, Gln, or Lys, Xaa₈ is Ser or Trp, Xaa₉ is His or Gln, and Xaa₁₀ is Lys or Asn; Formula IV
Trp-Xaa₁₁-Ser-Xaa₁₂-Arg-Val-Xaa₁₃(SEQ ID NO: 8), wherein Xaa₁₁ is Ala or Gly, Xaa₁₂ is Thr or Lys, and Xaa₁₃ is Ser or Pro; and Formula V
Xaa₁₄-Gln-Ser-Tyr-Ser-Xaa₁₅-Pro-Xaa₁₆-Thr (SEQ ID NO: 9), wherein Xaa₁₄ is Gly, Ala, or Gln, Xaa₁₅ is Arg, His, Ser, Ala, Gly, or Lys, and Xaa₁₆ is Leu, Tyr, Phe, or Met. Formula VI

13. The method of claim 12, wherein,
the CDR-H1 a polypeptide having an amino acid sequence of SEQ ID NO: 22, 23, or 24; the CDR-H2 is a polypeptide having an amino acid sequence of SEQ ID NO: 25 or 26.; the CDR-H3 is a polypeptide having an amino acid sequence of SEQ ID NO: 27 or 28; the CDR-L1 is a polypeptide having an amino acid sequence of SEQ ID NO: 29, 30, 31, 32, 33, or 71; the CDR-L2 is a polypeptide having an amino acid sequence of SEQ ID NO: 34, 35, or 36; or the CDR-L3 is a polypeptide having an amino acid sequence of SEQ ID NO: 13, 14, 15, 16, or 37.

14. The method of claim 11, wherein the anti-c-Met antibody or antigen-binding fragment thereof comprising:
a heavy chain variable region comprising at least one heavy chain complementarity determining region (CDR) selected from the group consisting of CDR-H1 having an amino acid sequence of SEQ ID NO: 1, CDR-H2 having an amino acid sequence of SEQ ID NO: 2, and CDR-H3 having an amino acid sequence of SEQ ID NO: 3; and
a light chain variable region comprising at least one light chain CDR selected from the group consisting of CDR-L1 having an amino acid sequence of SEQ ID NO: 7, CDR-L2 having an amino acid sequence of SEQ ID NO: 8, and CDR-L3 having an amino acid sequence of SEQ ID NO: 9, wherein SEQ ID NOS: 7 to 9 are respectively represented by Formula IV to VI below:
Lys-Ser-Ser-Xaa₇-Ser-Leu-Leu-Ala-Xaa₈-Gly-Asn-Xaa₉-Xaa₁₀-Asn-Tyr-Leu-Ala (SEQ ID NO: 7), wherein Xaa₇ is His, Arg, Gln, or Lys, Xaa₈ is Ser or Trp, Xaa₉ is His or Gln, and Xaa₁₀ is Lys or Asn; Formula IV
Trp-Xaa₁₁-Ser-Xaa₁₂-Arg-Val-Xaa₁₃(SEQ ID NO: 8), wherein Xaa₁₁ is Ala or Gly, Xaa₁₂ is Thr or Lys, and Xaa₁₃ is Ser or Pro; and Formula V
Xaa₁₄-Gln-Ser-Tyr-Ser-Xaa₁₅-Pro-Xaa₁₆-Thr (SEQ ID NO: 9), wherein Xaa₁₄ is Gly, Ala, or Gln, Xaa₁₅ is Arg, His, Ser, Ala, Gly, or Lys, and Xaa₁₆ is Leu, Tyr, Phe, or Met. Formula VI

15. The method of claim 14, wherein the anti-c-Met antibody or antigen-binding fragment thereof comprises the light chain variable region comprises at least one light chain CDR selected from the group consisting of CDR-L1 having an amino acid sequence of SEQ ID NO: 10 or 71, CDR-L2 having an amino acid sequence of SEQ ID NO: 11, and CDR-L3 having an amino acid sequence of SEQ ID NO: 13, 14, 15, or 16.

16. The method of claim 14, wherein the anti-c-Met antibody or antigen-binding fragment thereof comprises the heavy chain variable region has an amino acid sequence of SEQ ID NO: 17, and the light chain variable region has an amino acid sequence of SEQ ID NO: 18, 19, 20, 21, or 72.

17. The method of claim 11, wherein the anti-c-Met antibody or antigen-binding fragment thereof comprises:
a heavy chain comprising an amino acid sequence from 18^{th} to 462^{nd} of SEQ ID NO: 62, an amino acid sequence from 18^{th} to 461^{st} of SEQ ID NO: 64, or amino acid sequence from 18^{th} to 460^{th} of SEQ ID NO: 66, and a light chain comprising an amino acid sequence from 21^{st} to 220^{th} of SEQ ID NO: 68;
a heavy chain comprising an amino acid sequence from 18^{th} to 462^{nd} of SEQ ID NO: 62, an amino acid sequence from 18^{th} to 461^{st} of SEQ ID NO: 64, or amino acid sequence from 18^{th} to 460^{th} of SEQ ID NO: 66, and a light chain comprising an amino acid sequence from 21^{st} to 220^{th} of SEQ ID NO: 70; or
a heavy chain comprising an amino acid sequence from 18^{th} to 462^{nd} of SEQ ID NO: 62, an amino acid sequence from 18^{th} to 461^{st} of SEQ ID NO: 64, or amino acid sequence from 18^{th} to 460^{th} of SEQ ID NO: 66, and a light chain comprising an amino acid sequence of SEQ ID NO: 73.

18. The method of claim 11, wherein the anti-c-Met antibody or antigen-binding fragment thereof comprises an amino acid sequence of SEQ ID NO: 71, 72, or 73.

19. The method of claim 11, wherein the antibody or antigen-binding fragment is a monoclonal antibody, a mouse-derived antibody, a mouse-human chimeric antibody, or a humanized antibody,

20. The method of claim 11, wherein the antigen-binding fragment is scFv, (scFv)₂, Fab, Fab′, or F(ab′)₂.
